SEQUENCE LISTING (36 sequences)

SEQ ID NO: 1 - DNA, 1312 bp, Solanum lycopersicum
  source 1..1312: mol_type = genomic DNA
ttctagatggttctgttactaaatcactttcaactcactttgttgattcctagtctaaaagaattatcattgtgaaaaatcagtaggatattatataaccactcagaattaacacaatgaaaatttgacttgctatgatttgatgccgtaacatcaggtattctagttattgtggcctaccattgtttcattcagttccttgagcttacattgtgactttctatattatagagaacatttacagtatactatgtttattttgggttttccaaaaatgttgttttcatttaatttaccaagatgttgttttcttttgtagaggaatgtgttggatgagaaaataaagcgcaaggaagaacaagaggaaatgaaggtacacttttttgtgtcccattgggtatgaagattactcaaaacactcttgactacggatctattaacacaagatttggttctgtaggcagaattacaagctatgcgtgaagctgcccagtggagacgtttgcaaggttggtccattttgctaaamgtgctttatcatacatttattctactatcttgctcaaattttatgtgattggtacctaatgttttattcaactttgctattgcttaattgtctgtgaactccatctatgttggttagactcttgccaaacccatgtcaatccgacacaattcgggtgtggatgtgtgaagatgcaaataacagaaggaaatcaaaaagtagaaatagaataagaacgaaaggaagaaagtaatcaacccaagcaagtatacttgaacccactaattagcattcaattactagaatgaagtagaggaaaagaaatgaactcatatgtctatacttactagaagaattcgagagaggttcaattaaactggattgagaaaattccttaaagggctctgtcaagggagctcttcatcaagtaatttgccttagtatacctttcatgattttataatccccaaagtactcttctcacttaaagcctaactccactttcttttacttttttatgcctcattgcttggactacactatggtgcatgtgactagacaatagaaattctatttgcagcacaaacaataatcttgtgtaggcgtgtaaggtctgcgtacaccgactacaccccaccctgcccagaccccacttgtaggatcacactgggtatgttgttgttgtgttgttccgggtatgtgattgtttcctttgtcatgctccacacctttgaaggatatatggttccatctatcgtttgtggtactagatagtccatttctgatgaataaaacatacgaaga

SEQ ID NO: 2 - DNA, 199 bp, Solanum lycopersicum
  source 1..199: mol_type = genomic DNA
gagttattctctcacctgccctgaaatgtgtatgtatgaatacatttagggatgtcaacttcaattcataatttcaattctgaattctccaaaatttargatttcaaattccaatattttttttcaaatataaaacttgaccaataagattatattctgcacaattaatgtttatcttattaactatttatatttatga

SEQ ID NO: 3 - DNA, 1968 bp, Solanum lycopersicum
  source 1..1968: mol_type = genomic DNA
gagctcctcctggaggtgcatgcacagttattggaagtacccactcacatttctctctaccctctaccaaaagaggatgttcatatctgatacatcatgcatgaagtattacagaaacaaagagggataaattcgtgaatttagtctatttgaagaagaaaataccaaattacgaaggaaaatctataaatagaatcagatagatgaatcaggacatggagaacaaattgaggggagaaaagaacaccaagattacaattatggtgcatttgatgagagaccataaagttccttcccttaccatattttctttacaattgaatcgatatatctaagtgaagcaaatcaagggcatgcttcaaccttccatgattcactagaaataattatgtcaagagagttccactttaaaatagagattggaagttcaatcatgacaaatcgcaaggtgtcttctgattttccngtttgatggaagtggtaccaacagcaaaaattaaatattgatagcgggagagtatttcctgttaaaggtttctagcataagagtcatcataacaatgctgagtatcctagtttattgtgagaagatggagactcatcgttatcagcttatgattaggacggtggttcatgatgcattgcccaaataattttagaaaaagaggcaaaagattgtcagtcagagctacttaagttcgaatgaataagtgatagaatcttcttctccatgtgagtttcctagcacatttgacatctatttgttccgaagggttataagtcatgwagaggtgagcgtttagcctcttgatgatttacctggactaaaatggagatacgtaattaatgggctgcaattttattcaacagcttacatattgttcacggattcgcaggcatgaagaattggaaggttgaagcagaaaaaaattgtgcacaagatccctgtcaaaatttcttaaagtccctcatatttgatgggaataaagaagaaccgaagtagatgatgagctgatatacttctaaatccttttgatctgatgatctttttcagcttttaagatagtctgcaagcgtcttttgtatgatacttttccctgtaatgtactttaccctccccttttttgcaccctcttattggaaactccaaatcttcgatcaatgaattatgcagcgacaaaatattcttgttacatgagcaagtacataccttgacaagtcagcattcttgggagtggtaaagaattcaaatcgaagagcccattgtacggagacatagtgagtggaaaaagacataggaccatccatcggaatggaaaaaaggaagcttgtctgaattaaatcagcaaccacctcatgatgatcactatgaacctgaataaatcaaaaagccttggtgactaatcaccaaatgaaggggctcaagtgtttatctcttctaaataaatccaccaaaacacttctgtcaaatgcaaagagttgtaaagaaatcatcaacaggtaaagaataccttcgtaatagaaggagcatgtctccgtgaaggatgtacatagcgtcggctgatagtttcggtcatctccagtgtaattgaaagctagaaggcaacaaaaggatcaataaactccagaaagcataaactaatgaataaaccacatgtttaaggcttattacaagcatgaactttgatgtataggactacctcaaggcatcttctagaaccctcttcatgaaaaaaagtcagggttccacctatctgttgccaaagaataaattgaaggtataagttcccaaccagtcaggtgcaaataggcattggtaatagtactaatgaaaggaccaatacactgaataattcaattaccatatcactgaagtaatatgtcgactcagaattttttggagaaaatcttagaagcacttggtcatccagtctaatgttgtaagatctccctcgaataaacccttct

SEQ ID NO: 4 - DNA, 101 bp, Solanum lycopersicum
  source 1..101: mol_type = genomic DNA
tacgagccgtctcttgatgcttttctaccatagattttgatcgtctcgctktattatactgtttcaccagttcagcatccacacttgatctactcttttct

SEQ ID NO: 5 - DNA, 201 bp, Solanum lycopersicum
  source 1..201: mol_type = genomic DNA
ttgctggacaatacgatttaaggatactagacatgtttatgccctctaaatataatgactgtctaagttgctctgtcagtaaagcttagttggacccaaaytctattctcagtaatgcttagttggacccaaattctattttccttgacttgccatatttttgggctcggtcagtggcagatatctcaaggcctctccttt

SEQ ID NO: 6 - DNA, 201 bp, Solanum lycopersicum
  source 1..201: mol_type = genomic DNA
ttgcatattatgttgttggacataagactagtcactacaaaaaacatatgaagtcataagttgctttgaattctgctcaacgttttatttggttaacgttwgtcttaattgttttggtaggttaaattttggattctcactgaagatgtgtgacttatgaaataaaagttgttcagagagtacgttcgtagagtacttttg

SEQ ID NO: 7 - DNA, 201 bp, Solanum lycopersicum
  source 1..201: mol_type = genomic DNA
tctcccagtccattttggaagaagagtcgcagtgatttgttcaattatgttttgaatttantaaaagtatcttccaatgttgttttgggtttcaattggakttgtgaattacatgttcatttattttttttagaaaggtttcaagcaacatgtgcaatgagataattgttaaataatcttatcagtggatagtttaattat

SEQ ID NO: 8 - DNA, 201 bp, Solanum lycopersicum
  source 1..201: mol_type = genomic DNA
aaaacacactgcatgaaagtgttatttaaagaaatgggagagagtacaagataagtgagactcgaggtcttcaaggcattgatagattgaaaagagaatcwggtcccattcatttgctttttaaattgctcagaacagatttggtcttaagtttaaagcttaatacagggagattcggtgaagtagcaaccttttgatgag

SEQ ID NO: 9 - DNA, 187 bp, Solanum lycopersicum
  source 1..187: mol_type = genomic DNA
tgatatacatgcttgctagctggagcttcactagctcttattataatgatggtatcgncagttgactttcgacgtaatgaccaaatctatgttacattatyggaaaatgacattcattcaatcataatataaaatacaaaatatttaatcatgaagttttctagctcgacataacttaggaattaat

SEQ ID NO: 10 - DNA, 184 bp, Solanum lycopersicum
  source 1..184: mol_type = genomic DNA
agaaaatctaatcaaatggaatttcttagttggaattatgactatatttctacctgcatttgatgattcatgaaattttccctaataatgtgtagctactrtccgggaggataaattacgatcgaaatttcaaaaaantatttttttattattgcattttatttattcctaaactatatccttt

SEQ ID NO: 11 - DNA, 1001 bp, Solanum lycopersicum
  source 1..1001: mol_type = genomic DNA
atagaaagccaaagcaatacctctattgcacactatattaactaactataattaatatagatttcaatcacagtccacacaaataactctaactatactcattgttttttctcacagcaaagacttgatgcgttccttcatagattagaaacaaatctacaagttcactaaaacccaagttttcactaacaacacttaaaggatacactcgataactctatcaggtctcttaaggttgttgaaggcttagagtttttccaagacaaaacacaccttttcatatttttgcttttaatgtcttgactaagttttgtcatgtcaaatgaggatatattatcctgtgaacaaaaaaatttagtgtttactgattggtttagtacattgttctcgtttcttatgctcaccaaccacgccatagaggaccatcctttatagttatctatttgacttaagatacctgaatatagtgatagaatcagaattttcgccaaggatgttcatagttaggacatgtaaaacaataaacattgctagtcatctttttggttgacattttcatctagacgtaattgattttgattttgggrcgccaaacttgttttggctactctaaaaatatttttccaaactcattgaactccaaattgaaatagtagtcaagtataccgaacttattaaacatcatccacaatacacaaaatagttggtcgatactcaaataacaaagtagcttttaaaataaaacttaagatattttcaacacatgataagaaatctaaactaaaggaaaaaaagaattaagaaatatcgtttcacacaataatggcggtaatgattaaagcttccaatttcttaaaacttgcaaatatgtattgccaactccgctgagtttgcgcaatttcgagttcgtgtgagtgagttatgacctttggtaattggactgttggaataaggaaagtccaatccagattttagaaaggtagtttggtgttttccttacc

SEQ ID NO: 12 - DNA, 301 bp, Solanum habrochaites
  source 1..301: mol_type = genomic DNA
nnnnnnnnnnnnnnnnnnnnnnnntataggncactttttttttctctatatgccactaataagaatngaatcatgtttttttnnnnnntnggtatgattttgatatannnnnnnnnnnttttnnatagaantataaagtagaaattgacasataagattcaatanaatccaatctatatttcaatattttggagcaattcatgagatcntatattctttgttactataatattttacaattttaaaaataattntatttatataatttnnnantcctcacataattaataattacatacaa

SEQ ID NO: 13 - DNA, 301 bp, Solanum habrochaites
  source 1..301: mol_type = genomic DNA
tttgtgactttcatacctaaaattgttaggtgtttatataaccacccctgaantataccatgaaattattgtgcnaggtggcctaantngnaatttaatagggctgaattaagatttttgnagtnaattaaagaaaananttttttaattyatcctgaataagcntgctatttgngnggtttcagaaatatcggtagngccggtacntgggcnaataaaaattgattaaaaatataatnnnnnnnnnanantaaanttaaaattaaagagaatccaaactcaaaacaattaggttaatatt

SEQ ID NO: 14 - DNA, 301 bp, Solanum habrochaites
  source 1..301: mol_type = genomic DNA
taacttnaaantttctattttattcttaatngaangnattatagatagcaancaaatgtctaagtttgttttagaccataaattcaaaagtatttnnnnnnttttcttaaattttattatgtcaaancaaacgtgtcatataaatatgacyagaangaggattatagaaatgtttcgctataactaattaccctttncctctcaancgtttatcgtacnttttttgtnnctttaaaaagatntctctttncttatttgacaantctttaattntantttnntttnntcatttgacatatgt

SEQ ID NO: 15 - DNA, 2940 bp, Solanum habrochaites
  source 1..2940: mol_type = genomic DNA
atggctgaagctttccttcaaattatgttagagaatctgacttgtttcatccaaggggaacttggattgattcttggttttaaggatgagttcgaaaagcttcaaagcacgtttactacaatccaagctgtggtacaagatgctcagttgaagcaattgaaggacaaggcaattgaaaattggttgcagaaactcaatggtgctgcatatgaagctgatgacatcttggacgaatgtaaaactgaggcaccaattatacagaagaagaataaatatgggtgttatcatccaaacgttatcactttccgtcgcaagattgggaaaaggatgaaaaagattatggagaaactagatgcaattgcagcggaacgaattaagtttcatttggatgaaaggactatagagagacaagttgctacacgccaaacaggttttgttttaaatgaaccacaagtttatggaagagacaaagataaggatgagatagtgaaaatcctgataaacaatgcccaaacactttcagtcctcccaatacttggtatggggggactaggaaagacgacccttgcccaaatggtcttcaatgatcagagagtaattgaacatttccatcccaaaatatggatttgtgtctcggaagattttaatgaaaagaggttgataaagaaaattgtagaatctattgaagaaaagtcacttggtgacatggacttggctccacttcaaaagaagcttcaggacttgctgaatggaaaaaaatatttgcttgtcttagatgatgtttggaatgaagatcaagataagtgggctaagttaagacaagtcttgaaggctggagcaagtggtgcttatgttctaaccactacccgtcttgaaaaggttggatcaatcatggggacattgcaaccatatgaattgtcaaatttgtctcaagaagattgttggttgttgttcatgcaatgtgcatttgggcaccaagaagaaatgaatcttaatctagtggctatcggaaaggtgattgtgaaaaaatgtggtggtgtgcctctagcagctaaaactcttggaggtattttgcgcttcaagagagaagaaagacagtgggaacatgtgagagatagtgagatttggaatttacctcaagatgaaagttctattctgcctgccctgagacttagttaccatcaccttccacttgatttgagacaatgcttttcatattgtgcagtattcccaaaggataccaaaatggaaaaggaaaatctaatctctctctggatggcacatggttttcttttatcaaaaggaaacttggagctagaggatgtaggtaatgaagtatggaatgaattatacttgaggtcttttttccaagagattgaagttcaatatgatcgaacttatttcaagatgcatgatctcattcatgatttggcaacatctctattttcagcaagcacatcaagcagcaatatccgagaaataaatgtagaaggttacctacatatgatgtcgattggtttcgcaaaagtggtgtcttcttactctcctcctcacttgcaaaagtttgtctcattgagggttcttaatctaagttccatgggacttaagcagttaccgtcctccattggagatctagtacatttaagatacttgaacctctctctcaataacatgcgtactcttccaaagcagttatgcaagcttcaaaatctgcagactcttaatgtagagtattgctggtcactttgttgttttccaaaagaaacaagtaaacttggtagtctccgaaatctcttacttgatggttgcgatggattggattctatgccaccaaggataggatctttgacatgccttaagactctaagtttatttgttattggcgagagaaaagattctctacttggtgaattacgaaacctgaatctgtatgggtcaattgaaatcacgcatcttgagagagtgaagaatgatagggatgcaaaagaagccaatttatctgcaaaagaaaatctgcattctttaagcatgagatgggaaggaccacatagatatgaatcagaagaagttgaagtgcttgaatccctcaaaccacactccaatgtgacttgtttaacaatcactggcttcagaggaatccgtctcccagagtggatgaatcactcagttttgaaaaatgttgtctctattgcaattagaggttgtgaaaactgctcatgcttaccaccgtttggtgatctgccttgtctagaaagtctagagttacggagtgggtctgcggaagtggagtatgttgaagattctggattcccaacaagaagaaggtttccatctatgagaaaacttactatagaaaattttgataatctgaaaggattgctgaaagaggcaggagaagagcaattccccgtgcttgaagagttgacaattagatgttgtcctgtgtttgttattccgaccctttcttctgtcaagaaattggtagttcatgggaacaagtcagatgcaatagttttgaggtccatatataatcttagggctcttacttccctcaacattagccataacttcacagctacttcgctcccagaagagatgttcaaaagccttgcaaatctcaaatacttggaaatcgctttcatctccaatctcaaagagctgccaaacagcctggctagtctcaatgctttgaagcatctgtttattaattgttgttttgcactagagagtctccccgaggaagcggtgaaaggtttaacttcactcacacagttatccataacatactgtaagaggctaaaatgtttaccagagggattgcagcaactaacaaatttatcagttaggtattgtccaacactggccaagcgatgtgagaagggaataggacaagactggtacaaaattgctcacattcctcatctgctgattactgattag

SEQ ID NO: 16 - DNA, 2940 bp, Solanum habrochaites
  source 1..2940: mol_type = genomic DNA
atggctgaagctttccttcaaattatgttagagaatctgacttgtttcatccaaggggaacttggattgattcttggttttaaggatgagttcgaaaagcttcaaagcacgtttactacaatccaagctgtggtacaagatgctcagttgaagcaattgaaggacaaggcaattgaaaattggttgcagaaactcaatggtgctgcatatgaagctgatgacatcttggacgaatgtaaaactgaggcaccaattatacagaagaagaataaatatgggtgttatcatccaaacgttatcactttccgtcgcaagattgggaaaaggatgaaaaagattatggagaaactagatgcaattgcagcggaacgaattaagtttcatttggatgaaaggactatagagagacaagttgctacacgccaaacaggttttgttttaaatgaaccacaagtttatggaagagacaaagataaggatgagatagtgaaaatcctgataaacaatgcccaaacactttcagtcctcccaatacttggtatggggggactaggaaagacgacccttgcccaaatggtcttcaatgatcagagagtaattgaacatttccatcccaaaatatggatttgtgtctcggaagattttaatgaaaagaggttgataaaggaaattgtagaatctattgaagaaaagtcacttggtgacatggacttggctccacttcaaaagaagcttcaggacttgctgaatggaaaaaaatatttgcttgtcttagatgatgtttggaatgaagatcaagataagtgggctaagttaagacaagtcttgaaggctggagcaagtggtgcttatgttctaaccactacccgtcttgaaaaggttggatcaatcatggggacattgcaaccatatgaattgtcaaatttgtctcaagaagattgttggttgttgttcatgcaatgtgcatttgggcaccaagaagaaatgaatcttaatctagtggctatcggaaaggtgattgtgaaaaaatgtggtggtgtgcctctagcagctaaaactcttggaggtattttgcgcttcaagagagaagaaagacagtgggaacatgtgagagatagtgagatttggaatttacctcaagatgaaagttctattctgcctgccctgagacttagttaccatcaccttccacttgatttgagacaatgcttttcatattgtgcagtattcccaaaggataccaaaatggaaaaggaaaatctaatctctctctggatggcacatggttttcttttatcaaaaggaaacttggagctagaggatgtaggtaatgaagtatggaatgaattatacttgaggtcttttttccaagagattgaagttcaatatgatcgaacttatttcaagatgcatgatctcattcatgatttggcaacatctctattttcagcaagcacatcaagcagcaatatccgagaaataaatgtagaaggttacctacatatgatgtcgattggtttcgcaaaagtggtgtcttcttactctcctcctcacttgcaaaagtttgtctcattgagggttcttaatctaagttccatgggacttaagcagttaccgtcctccattggagatctagtacatttaagatacttgaacctctctctcaataacatgcgtactcttccaaagcagttatgcaagcttcaaaatctgcagactcttaatgtagagtattgctggtcactttgttgttttccaaaagaaacaagtaaacttggtagtctccgaaatctcttacttgatggttgcgatggattggattctatgccaccaaggataggatctttgacatgccttaagactctaagtttatttgttattggcgagagaaaagattctctacttggtgaattacgaaacctgaatctgtatgggtcaattgaaatcacgcatcttgagagagtgaagaatgatagggatgcaaaagaagccaatttatctgcaaaagaaaatctgcattctttaagcatgagatgggaaggaccacatagatatgaatcagaagaagttgaagtgcttgaatccctcaaaccacactccaatgtgacttgtttaacaatcactggcttcagaggaatccgtctcccagagtggatgaatcactcagttttgaaaaatgttgtctctattgcaattagaggttgtgaaaactgctcatgcttaccaccgtttggtgatctgccttgtctagaaagtctagagttacggagtgggtctgcggaagtggagtatgttgaagattctggattcccaacaagaagaaggtttccatctatgagaaaacttactatagaaaattttgataatctgaaaggattgctgaaagaggcaggagaagagcaattccccgtgcttgaagagttgacaattagatgttgtcctgtgtttgttattccgaccctttcttctgtcaagaaattggtagttcatgggaacaagtcagatgcaatagttttgaggtccatatataatcttagggctcttacttccctcaacattagccataacttcacagctacttcgctcccagaagagatgttcaaaagccttgcaaatctcaaatacttggaaatcgctttcatctccaatctcaaagagctgccaaacagcctggctagtctcaatgctttgaagcatctgtttattaattgttgttttgcactagagagtctccccgaggaagcggtgaaaggtttaacttcactcacacagttatccataacatactgtaagaggctaaaatgtttaccagagggattgcagcaactaacaaatttatcagttaggtattgtccaacactggccaagcgatgtgagaagggaataggacaagactggtacaaaattgctcacattcctcatctgctgattactgattag

SEQ ID NO: 17 - DNA, 2933 bp, Solanum habrochaites
  source 1..2933: mol_type = genomic DNA
atggctgaagctttccttcaaattatgttagagaatctgacttgtttcatccaaggggaacttggattgattcttggttttaaggatgagttcaaaaagcttcaaagcacgtttactacaatccaagctgtggtacaagatgctcagttgaagcaattgaaggacaaggcaattgaaaattggttgcagaaactcaatggtgctgcatatgaagctgatgacatcttggacgaatgtaaaactgaggcaccaattatacagaagaagaataaatatgggtgttatcatccaaacgttatcactttccgtcacaagattgggaaacggatgaaaaagattatggagaaactagatgcaattgcagcggaacgaattaagtttcatttggatgaaaggactatagagagacaagttgctatacgccaaacaggttttgttttaaatgaaccacaagtttatggaagagacaaagataaggatgagatagtgaaaatcctgataaacaatgcccaaacactttcagtcctcccaatacttggtatggggggactaggaaagacgacccttgcccaaatggtcttcaatgatcagagagtaattgagcatttccatcccaaaatatggatttgtgtctcggaagatttaatgaaaagaggttgataaaggaaattgtagaatctattgaagaaaagtcacttggtgacatggacttggctccacttcaaaagaagcttcgggacttgttgaatggaaaaagatacttgcttgtcttggatgatgtttggaatgaagatcaagataagtgggctaagttaagacaagtcttgaaggttggagcaagtggtgcttctgttctaaccactactcgtcttgaaaaggttggatcaattatggcaacattgcaaccatatgaattgtcaaacttttctcaagaagattgttggttgttgttcatgcaacgtgcatttgggcactaagaagaaataaatcttaatcttgtggctatcggaaaggtgattgtgagaaaatgtggtggtgtgcctctagcagctaaaactcttggaggtattttgcgcttcaagagagaagaaagacagtgggaacatgtgagagatagtgagatttggaaattgcctcaagaagaaagttctattctgcctgccctgagacttagttaccatcaccttccacttgatttgagacaatgcttttcatattgtgcagtattcccaaaggataccaaaatggaaaaggaaaatctaatctctctgtggatggcacatggttttcttttatcaaaaggaaacttggagctagaggatgtaggtaatgaagtatggaatgaattatacttgaggtcttttttccaagagattgaagttaaatatgatcgaacttatttcaagatgcatgatctcattcatgatttggcaacatctctattttcagcaagcacatcaagcagcaatatccgagaaataaatgtagaaggttacctacatatgatgtcgattggtttcacaaaagtggtgtcttcttactctcctcctcacttgcaaaagtttgtctcattgagggttcttaatctaagttccgtgggacttaagcagttaccgtcctccattggagatctagtacatttaagatacttgaacctctctctcaataacatgcgtactcttccaaagcagttatgcaagcttcaaaatctgcagactcttaatgtagagtattgctggtcactttgttgtttgccaaaagaaacaagtaaacttggtagtctccgaaatctcttacttgatggttgcgatggattggattctatgccaccaaggataggatctttgacatgccttaagactctaagtttctttgttattggcgagagaaaagattctctacttggtgaattacgaaacctgaatttgtatgggtcagttgaaatcacgcatcttgagagagtgaagaatgatagggatgcaaaagaagccaatttatctgcaaaagaaaatctgcattctttaagcatgagatggaaaaaaccacatagatatgaatcagaagaagttgaagtgcttgaatccctcaaaccacaccctaatttgacttctttactaatcactggcttcagaggattccgtcttccaaagtggatgaatcactcagttttgaaaaatgttgtctctattgcaattagaggttgtgaaaactgctcatgcttaccaccgtttggtgatctgccttgtcttgaaagtctagagttaggagatgggtctgcggaactgaagtatgttgaagattctggattccctacaagaagaaggtttccatctctgagaaaacttattatagtcaattttgataatctgaaaggattgttgaaagaggcaggagaagagcaattccccgtgcttgaagagatgacaattagctggtgtcctgtgcttgttattccgaccctttcttctgtcaagaaattggtagtttatcggaacatgtcagatgcaataggtttgaggtccatatataatcttagggctcttacttccctcaacattagccataacttgacagctacttcgctcccagaagagatgttcaaaagccttgcaaatctcaaatacttggaaatctctttcatcttcaatctcaaagagctgccaaacagcctggctagtctcaatgctttgaagcatctgaaaattgaatattgtgacgcactcgagagtctccccgaggaaggggtgaaaggtttaacttcactcacagaattatccataacaaattgtaagaggctaaaatgtttaccggagggattgcagcacctaacaaatttatcagttagggaatgtccaacactggccaagcggtgtgagaagggaataggacaagactggtacaaaattgctcacattcctcatctgcttattact

SEQ ID NO: 18 - protein, 979 aa, Solanum habrochaites
MAEAFLQIMLENLTCFIQGELGLILGFKDEFEKLQSTFTTIQAVVQDAQLKQLKDKAIENWLQKLNGAAYEADDILDECKTEAPIIQKKNKYGCYHPNVITFRRKIGKRMKKIMEKLDAIAAERIKFHLDERTIERQVATRQTGFVLNEPQVYGRDKDKDEIVKILINNAQTLSVLPILGMGGLGKTTLAQMVFNDQRVIEHFHPKIWICVSEDFNEKRLIKKIVESIEEKSLGDMDLAPLQKKLQDLLNGKKYLLVLDDVWNEDQDKWAKLRQVLKAGASGAYVLTTTRLEKVGSIMGTLQPYELSNLSQEDCWLLFMQCAFGHQEEMNLNLVAIGKVIVKKCGGVPLAAKTLGGILRFKREERQWEHVRDSEIWNLPQDESSILPALRLSYHHLPLDLRQCFSYCAVFPKDTKMEKENLISLWMAHGFLLSKGNLELEDVGNEVWNELYLRSFFQEIEVQYDRTYFKMHDLIHDLATSLFSASTSSSNIREINVEGYLHMMSIGFAKVVSSYSPPHLQKFVSLRVLNLSSMGLKQLPSSIGDLVHLRYLNLSLNNMRTLPKQLCKLQNLQTLNVEYCWSLCCFPKETSKLGSLRNLLLDGCDGLDSMPPRIGSLTCLKTLSLFVIGERKDSLLGELRNLNLYGSIEITHLERVKNDRDAKEANLSAKENLHSLSMRWEGPHRYESEEVEVLESLKPHSNVTCLTITGFRGIRLPEWMNHSVLKNVVSIAIRGCENCSCLPPFGDLPCLESLELRSGSAEVEYVEDSGFPTRRRFPSMRKLTIENFDNLKGLLKEAGEEQFPVLEELTIRCCPVFVIPTLSSVKKLVVHGNKSDAIVLRSIYNLRALTSLNISHNFTATSLPEEMFKSLANLKYLEIAFISNLKELPNSLASLNALKHLFINCCFALESLPEEAVKGLTSLTQLSITYCKRLKCLPEGLQQLTNLSVRYCPTLAKRCEKGIGQDWYKIAHIPHLLITD

SEQ ID NO: 19 - protein, 979 aa, Solanum habrochaites
MAEAFLQIMLENLTCFIQGELGLILGFKDEFEKLQSTFTTIQAVVQDAQLKQLKDKAIENWLQKLNGAAYEADDILDECKTEAPIIQKKNKYGCYHPNVITFRRKIGKRMKKIMEKLDAIAAERIKFHLDERTIERQVATRQTGFVLNEPQVYGRDKDKDEIVKILINNAQTLSVLPILGMGGLGKTTLAQMVFNDQRVIEHFHPKIWICVSEDFNEKRLIKEIVESIEEKSLGDMDLAPLQKKLQDLLNGKKYLLVLDDVWNEDQDKWAKLRQVLKAGASGAYVLTTTRLEKVGSIMGTLQPYELSNLSQEDCWLLFMQCAFGHQEEMNLNLVAIGKVIVKKCGGVPLAAKTLGGILRFKREERQWEHVRDSEIWNLPQDESSILPALRLSYHHLPLDLRQCFSYCAVFPKDTKMEKENLISLWMAHGFLLSKGNLELEDVGNEVWNELYLRSFFQEIEVQYDRTYFKMHDLIHDLATSLFSASTSSSNIREINVEGYLHMMSIGFAKVVSSYSPPHLQKFVSLRVLNLSSMGLKQLPSSIGDLVHLRYLNLSLNNMRTLPKQLCKLQNLQTLNVEYCWSLCCFPKETSKLGSLRNLLLDGCDGLDSMPPRIGSLTCLKTLSLFVIGERKDSLLGELRNLNLYGSIEITHLERVKNDRDAKEANLSAKENLHSLSMRWEGPHRYESEEVEVLESLKPHSNVTCLTITGFRGIRLPEWMNHSVLKNVVSIAIRGCENCSCLPPFGDLPCLESLELRSGSAEVEYVEDSGFPTRRRFPSMRKLTIENFDNLKGLLKEAGEEQFPVLEELTIRCCPVFVIPTLSSVKKLVVHGNKSDAIVLRSIYNLRALTSLNISHNFTATSLPEEMFKSLANLKYLEIAFISNLKELPNSLASLNALKHLFINCCFALESLPEEAVKGLTSLTQLSITYCKRLKCLPEGLQQLTNLSVRYCPTLAKRCEKGIGQDWYKIAHIPHLLITD

SEQ ID NO: 20 - DNA, 101 bp, unidentified
  source 1..101: mol_type = genomic DNA; note = Lycopersicon esculentum
atggcaatagtgaactgcagatacaactgaaattgcagaacacccttaaawatagaatcaatagaaagttgcaacaatatttgaatgatgaagcaacaaag

SEQ ID NO: 21 - DNA, 101 bp, unidentified
  source 1..101: mol_type = genomic DNA; note = Lycopersicon esculentum
agccattgtgattgtgtctgttgtacattaccaaaattctctagagaaagygatacacatgccagccctatcgatataaagcaacgcaaggtggattctgc

SEQ ID NO: 22 - DNA, 101 bp, unidentified
  source 1..101: mol_type = genomic DNA; note = Lycopersicon esculentum
aacaccaggtagagagcacagcgaaacaatggcctcaggaagatctacttygcgaagtgcagcaagccactccatacctccaccaggctttgatttcagtg

SEQ ID NO: 23 - DNA, 101 bp, unidentified
  source 1..101: mol_type = genomic DNA; note = Lycopersicon esculentum
tcaactgcaactttaacagctgattcaacttcttcttctttcgaaacatcrcattgaatgtaacgacctccaatagattcagctaaacttgtacctacttc

SEQ ID NO: 24 - DNA, 101 bp, unidentified
  source 1..101: mol_type = genomic DNA; note = Lycopersicon esculentum
gaggagctatcaacttcatagtcagattcagaaaatgattcagatgaggamgtggctgattcttcttgttttcttttcttccttctgctcgaactctctcc

SEQ ID NO: 25 - DNA, 101 bp, unidentified
  source 1..101: mol_type = genomic DNA; note = Lycopersicon esculentum
cagaaataatagaaaatcagaaagaaaaatcagctttctaaatggaaaagrcgatggcactatgtttgaagttttaagcaacttttctgaagtcccaaaag

SEQ ID NO: 26 - DNA, 160 bp, unidentified
  source 1..160: mol_type = genomic DNA; note = Lycopersicon esculentum
ctcctattgaacatcctgaaaacttgtgtctacatcatgagaagatgcaggccaattcrctcagtacatggaatgcacgagcatgttaggggaattctaacgcaaagcataagcttgatacttgaataaaagatgaaacatacttacttcttctcaaact

SEQ ID NO: 27 - DNA, 101 bp, unidentified
  source 1..101: mol_type = genomic DNA; note = Lycopersicon esculentum
ctcttggtgacaaaccactggctcaatttcttcgcgaagctaaagctatcrctgatgagcttgtcacggcaggcacacgtgtctcctgatgaattcaatgc

SEQ ID NO: 28 - DNA, 101 bp, unidentified
  source 1..101: mol_type = genomic DNA; note = Lycopersicon esculentum
cattactgttgagatatctcatcggcaacccctggagcttgcccacccgcktgtcctccaggatctgatttcagaaaggatgaatagtaactgtgtttcag

SEQ ID NO: 29 - DNA, 121 bp, unidentified
  source 1..121: mol_type = genomic DNA; note = Lycopersicon esculentum
caagaacccaacgacttcttcttctttgcttattgaaaaacttggttttgaaatgaaaggratcgagaaattggatactcagtggttctctactactaaaccttctcctgattttaagaaa

SEQ ID NO: 30 - DNA, 101 bp, unidentified
  source 1..101: mol_type = genomic DNA; note = Lycopersicon esculentum
tgatcgacaattcttgttgttgttgaaactctgcaagtgagagagggatgyatatagagaaaggatattggtaaaggacaattctagaagggtctagggaa

SEQ ID NO: 31 - DNA, 101 bp, unidentified
  source 1..101: mol_type = genomic DNA; note = Lycopersicon esculentum
gcaccagttatagtaatgtcctgcttctttcctgtacccttatcagtagcygtgacagaaagaataccgttggtgtcaatgtcgaacttcacttcaatctg

SEQ ID NO: 32 - DNA, 117 bp, Solanum lycopersicum
  source 1..117: mol_type = genomic DNA
ataggcttgcccttacttatttgatctttcaatttctgcagtatcacarttcttttctgcaaagtttctgctcaaggaagattaggcttctcatcaagtcttcatacaaattaaaag

SEQ ID NO: 33 - DNA, 301 bp, Solanum habrochaites
  source 1..301: mol_type = genomic DNA
tacaacttatttacatcccactggataattctcaaattgnaaagatctcttatttnatctattttgcaggtacacaattgtttcaacgggaggcactgcatctgctttggaaggtgctggtgtntctgtcaccaaagtggaagagctgacwcgtttccctgaaatggtaggaactcctgantaatgtttgaaaggtataactggaacttgccnngnnnnnnggtaaggcaatncctaaanactcccatctntccaagctttggtngatggtgctaccnggtatctgtgccggtggnaggta

SEQ ID NO: 34 - DNA, 301 bp, Solanum habrochaites
  source 1..301: mol_type = genomic DNA
gcntttctatgtggcggtggatgaggtatcatcgttacttctgnaacaccccaccccacccccggcataccacttccttttccaagtaattantggttgatcatcaatcaagcatatatncgtnttaaacaacatttggttangagaactracttgattttgtaagaagaacgaatagatttggtggggaannnnnnnnnnnnnncccnnaatagttggncncaaactatnaaatataataaatgcatccaaacataggtatattgtatctatagttatctaaaaagagcancaaatccta

SEQ ID NO: 35 - DNA, 301 bp, Solanum habrochaites
  source 1..301: mol_type = genomic DNA
gagtagttaattgtagtttggtgatttgatgtttgtcattcgattnttctcttttaattaatctgtctaattatatatattatattcaaattatcattgcatggaaatanaattataattatatatagtctcnaatcatgcnnancttacmcccacaattgggggcacaaatagacaaacattcagcttgcttanatgatgggattggctcaanttcagttcacatatacattattatattataatatcttataataaaataaaatataaaagatatatcacataaattattggattttga

SEQ ID NO: 36 - DNA, 301 bp, Solanum habrochaites
  source 1..301: mol_type = genomic DNA
aatncggtttanntatcngtctctctagaaataaggttcattcttttttaaanaaactaaaaatgnaagtaagacaataaattgaaacancgggagtacttgtaaaagtgaaaatccggtttagctactctagagatgcatttgatatagycaaaaagtacaagtttcagtagatagttgtgtgccctgtttagttgaagttgaaaatgttaattgaatgaattgccatncatttttttcatncttnataggataaatcaaacttatctcatcgtttcatttntattggagttgaacatgc

(19) United States
(12) Patent Application Publication
(10) Pub. No.: US 2026/0293829 A1
(43) Pub. Date: Oct. 1, 2026

(54) METHODS AND COMPOSITIONS FOR TOMATO BROWN RUGOSE FRUIT VIRUS RESISTANCE IN TOMATO

(71) Applicant: Seminis Vegetable Seeds, Inc., St. Louis, MO (US)

(72) Inventors: Johannes Hendrikus Maria Barten, Roquetas de Mar (ES); Mélissa Christine Cantet, Avignon (FR); Maria Leontine Hop, Wagenberg (NL); Benjamin C. Hunter, Zuid-Holland (NL); Maria Belen Salleres Neira, Almeria (ES); Antoon Stekelenburg, Houten (NL)

(21) Appl. No.: 19/635,138

(22) Filed: Mar. 31, 2026

Related U.S. Application Data
(60) Provisional application No. 63/781,786, filed on Apr. 1, 2025.
(60) Provisional application No. 63/805,779, filed on May 14, 2025.

(51) Int. Cl. A01H 1/00; A01H 5/08; A01H 6/82; C07K 14/415

(52) U.S. Cl. CPC A01H 1/126; A01H 5/08; A01H 6/825; C07K 14/415

(57) ABSTRACT

Tomato (Solanum lycopersicum) plants exhibiting resistance to resistance-breaking isolates of Tomato Brown Rugose Fruit Virus (ToBRFV) are provided, together with methods of producing, identifying, or selecting plants or germplasm with a ToBRFV resistance phenotype. Such plants include tomato plants comprising introgressed genomic regions conferring pest resistance.

### CROSS-REFERENCE TO RELATED APPLICATIONS

This application claims the priority of U.S. Provisional Appl. Ser. Nos. 63/781,786 and 63/805,779, filed Apr. 1, 2025 and May 14, 2025, respectively, the entire disclosures of which are incorporated herein by reference.

### FIELD OF THE INVENTION

The present invention relates to the field of plant breeding and, more specifically, to methods and compositions for producing tomato plants with desired resistance to Tomato Brown Rugose Fruit Virus (ToBRFV).

### INCORPORATION OF SEQUENCE LISTING

The sequence listing that is contained in the file named “SEMB060US_ST26.xml,” which is 54,197 bytes as measured in Microsoft Windows operating system and was created on Mar. 28, 2026, is filed electronically herewith and incorporated herein by reference.

### BACKGROUND OF THE INVENTION

Plant disease resistance is an important trait in plant breeding, particularly for production of food crops. Tomato Brown Rugose Fruit Virus (ToBRFV) belongs to the genus Tobamovirus, which also includes other pathogenic viruses such as Tobacco mosaic virus and Tomato mosaic virus. ToBRFV is an emerging and rapidly spreading RNA virus that primarily infects tomato plants, as well as peppers. Tomato plants infected with ToBRFV show mosaic symptoms on young leaves, including dark green bulges, narrowing, and deformation. Peduncles and calyces often become necrotic, preventing fruit production. Fruits develop yellow blotches, brown or black spots, and wrinkling. In peppers, infection causes puckered leaves with yellow mottling, stunted seedlings, and small yellow to brown rugose dots and necrotic blotches on fruits. Breaking isolates that overcome resistance conferred by known ToBRFV resistance sources have recently been reported. The ability of these breaking isolates to overcome resistance in tomato plants presents a significant threat to tomato production. The development of tomato varieties with effective levels of resistance to ToBRFV, including resistance-breaking ToBRFV isolates, is therefore increasingly important.

### SUMMARY OF THE INVENTION

In one aspect, the invention provides a Solanum lycopersicum plant comprising a Tm1 resistance gene on chromosome 2; a recombinant chromosomal segment on chromosome 11, wherein said chromosomal segment comprises a Tomato Brown Rugose Fruit Virus (ToBRFV) resistance allele; and a ToBRFV resistance gene on chromosome 8 encoding a ToBRFV resistance protein comprising a polypeptide sequence of SEQ ID NO:18 or 19; wherein the plant exhibits increased resistance to ToBRFV, including resistance-breaking ToBRFV isolates. In some embodiments, said ToBRFV resistance allele is further defined as: located within a chromosomal segment flanked by marker locus M1 (SEQ ID NO:1) and marker locus M3 (SEQ ID NO:3) on chromosome 11 in said plant; within a chromosomal segment on chromosome 11 comprising marker locus M2 (SEQ ID NO:2) or marker locus M11 (SEQ ID NO:11) in said plant; or said ToBRFV resistance gene on chromosome 8 is further defined as: located within a chromosomal segment flanked by marker locus M12 (SEQ ID NO:12) and marker locus M14 (SEQ ID NO:14) on chromosome 8 in said plant; or within a chromosomal segment on chromosome 8 comprising marker locus M13 (SEQ ID NO:13) in said plant. In certain embodiments, said ToBRFV resistance gene is encoded by a polynucleotide sequence comprising SEQ ID NO:16. In other embodiments, a representative sample of seed comprising said recombinant chromosomal segment has been deposited under NCMA Accession No. 202103011. In some embodiments, a representative sample of seed comprising the Tm1 resistance gene on chromosome 2, the recombinant chromosomal segment on chromosome 11, and/or the ToBRFV resistance gene on chromosome 8 encoding a ToBRFV resistance protein comprising a polypeptide sequence of SEQ ID NO:19 has been deposited under NCMA Accession No. 202504007. In some embodiments, said plant is homozygous for: the Tm1 resistance gene; the ToBRFV resistance allele; or the ToBRFV resistance gene. In further embodiments, said plant is homozygous for the Tm1 resistance gene, the ToBRFV resistance allele, and the ToBRFV resistance gene. In certain embodiments, said resistance-breaking ToBRFV isolate comprises an Asn to Lys substitution at position 82 of a ToBRFV movement protein. In specific embodiments, said plant comprises resistance to resistance-breaking ToBRFV isolate Vir353 (NCBI GenBank Accession OR760199). In some embodiments, said plant exhibits reduced ToBRFV-associated leaf symptoms when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19. In other embodiments, said plant exhibits reduced ToBRFV transmissibility when infected with resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19. In further embodiments, said plant or plant parts exhibit reduced ToBRFV titer when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19. In certain embodiments, the Solanum lycopersicum plant comprising a Tm1 resistance gene on chromosome 2; a recombinant chromosomal segment on chromosome 11, wherein said chromosomal segment comprises a ToBRFV resistance allele; and a ToBRFV resistance gene on chromosome 8 encoding a ToBRFV resistance protein comprising a polypeptide sequence of SEQ ID NO:18 or 19, exhibits increased resistance to resistance-breaking Tomato Brown Rugose Fruit Virus (ToBRFV) isolates during: a crop cycle greater than or equal to about 9, about 8, about 7, about 6, about 5, about 4, or about 3 months; under abiotic stress conditions; or under biotic stress conditions. In other embodiments, said resistance to ToBRFV comprises resistance to non-resistance-breaking ToBRFV isolates.

In another aspect, the invention provides a Solanum lycopersicum plant comprising a Tm1 resistance gene on chromosome 2; a recombinant chromosomal segment on chromosome 11, wherein said chromosomal segment comprises a Tomato Brown Rugose Fruit Virus (ToBRFV) resistance allele; and a ToBRFV resistance gene on chromosome 8 encoding a ToBRFV resistance protein comprising a polypeptide sequence of SEQ ID NO:18 or 19; wherein the plant exhibits broad-spectrum resistance to ToBRFV.

In another aspect, cells, seed, and plant parts comprising a Tm1 resistance gene on chromosome 2; a recombinant chromosomal segment on chromosome 11, wherein said chromosomal segment comprises a Tomato Brown Rugose Fruit Virus (ToBRFV) resistance allele; and a ToBRFV resistance gene on chromosome 8 encoding a ToBRFV resistance protein comprising a polypeptide sequence of SEQ ID NO:18 or 19 are provided.

In another aspect, methods are provided for selecting a tomato plant exhibiting increased resistance to resistance-breaking Tomato Brown Rugose Fruit Virus (ToBRFV) isolates, comprising, crossing a Solanum lycopersicum plant comprising a Tm1 resistance gene on chromosome 2; a recombinant chromosomal segment on chromosome 11, wherein said chromosomal segment comprises a ToBRFV resistance allele; and a ToBRFV resistance gene on chromosome 8 encoding a ToBRFV resistance protein comprising a polypeptide sequence of SEQ ID NO:18 or 19, with itself or with a second tomato plant of a different genotype to produce one or more progeny plants; and selecting a progeny plant comprising the Tm1 resistance gene on chromosome 2, the recombinant chromosomal segment on chromosome 11, and the ToBRFV resistance gene.

In further aspects, methods are provided for producing a tomato plant exhibiting increased resistance to resistance-breaking Tomato Brown Rugose Fruit Virus (ToBRFV) isolates, comprising introgressing into a plant a Tm1 resistance gene on chromosome 2, a recombinant chromosomal segment on chromosome 11, wherein said chromosomal segment comprises a ToBRFV resistance allele, or a ToBRFV resistance gene on chromosome 8 encoding a ToBRFV resistance protein comprising a polypeptide sequence of SEQ ID NO:18 or 19, wherein the tomato plant exhibits increased resistance to resistance-breaking ToBRFV isolates. In some embodiments, said method comprises introgressing into the plant: the Tm1 resistance gene on chromosome 2; the recombinant chromosomal segment on chromosome 11; the ToBRFV resistance gene on chromosome 8 encoding a ToBRFV resistance protein comprising a polypeptide sequence of SEQ ID NO:18 or 19; or a combination of any thereof. In other embodiments, said introgressing comprises: crossing a plant comprising the Tm1 resistance gene on chromosome 2, the recombinant chromosomal segment on chromosome 11, and the ToBRFV resistance gene on chromosome 8 with itself or with a second Solanum lycopersicum plant of a different genotype to produce one or more progeny plants; and selecting a progeny plant comprising the Tm1 resistance gene on chromosome 2, the recombinant chromosomal segment on chromosome 11, and the ToBRFV resistance gene on chromosome 8. In further embodiments, a sample of seed comprising said recombinant chromosomal segment has been deposited under NCMA Accession No. 202103011. In certain embodiments, selecting a progeny plant comprises detecting nucleic acids comprising marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), M12 (SEQ ID NO:12), M13 (SEQ ID NO:13), M17 (SEQ ID NO:35), or M14 (SEQ ID NO:14). Tomato plants obtainable by the methods disclosed herein are further provided. In further embodiments, the progeny plant is an F₂-F₆ progeny plant. In yet further embodiments, the method comprises backcrossing, marker-assisted selection, or assaying for said ToBRFV resistance.

In yet a further aspect, methods are provided controlling or preventing Tomato Brown Rugose Fruit Virus (ToBRFV) symptoms during crop production, wherein the ToBRFV symptoms are caused by resistance-breaking ToBRFV isolates, comprising: obtaining a population of Solanum lycopersicum plants comprising a ToBRFV resistance gene on chromosome 8 encoding a ToBRFV resistance protein comprising a polypeptide sequence of SEQ ID NO:19; growing the Solanum lycopersicum plants; and harvesting fruit of the Solanum lycopersicum plants. In some embodiments, the population of Solanum lycopersicum plants further comprise: a Tm1 resistance gene on chromosome 2; or a recombinant chromosomal segment on chromosome 11, wherein said chromosomal segment comprises a ToBRFV resistance allele. In other embodiments, the population of Solanum lycopersicum plants comprises: the ToBRFV resistance gene on chromosome 8 encoding a ToBRFV resistance protein comprising a polypeptide sequence of SEQ ID NO:19; the Tm1 resistance gene on chromosome 2; and the recombinant chromosomal segment on chromosome 11, wherein said chromosomal segment comprises a ToBRFV resistance allele.

In yet another aspect, methods are provided for improving tomato crop yield in an area comprising resistance-breaking Tomato Brown Rugose Fruit Virus (ToBRFV) isolates, the method comprising: growing a population of Solanum lycopersicum plants comprising a ToBRFV resistance gene on chromosome 8 encoding a ToBRFV resistance protein comprising a polypeptide sequence of SEQ ID NO:19; and harvesting fruit of the Solanum lycopersicum plants, wherein the tomato crop yield of said population of Solanum lycopersicum plants is increased as compared to the yield of a population of Solanum lycopersicum plants lacking the ToBRFV resistance gene. In some embodiments, the population of Solanum lycopersicum plants further comprise: a Tm1 resistance gene on chromosome 2; or a recombinant chromosomal segment on chromosome 11, wherein said chromosomal segment comprises a ToBRFV resistance allele. In other embodiments, the population of Solanum lycopersicum plants comprises: the ToBRFV resistance gene on chromosome 8 encoding a ToBRFV resistance protein comprising a polypeptide sequence of SEQ ID NO:19; the Tm1 resistance gene on chromosome 2; and the recombinant chromosomal segment on chromosome 11, wherein said chromosomal segment comprises a ToBRFV resistance allele. In certain embodiments, the crop yield of said population of Solanum lycopersicum plants is increased as compared to the yield of a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19. In other embodiments, the crop yield of said population of Solanum lycopersicum plants is increased as compared to the yield of a control plant comprising the Tm1 resistance gene, the ToBRFV resistance allele, and a ToBRFV resistance gene on chromosome 8 encoding a ToBRFV resistance protein comprising a polypeptide sequence of SEQ ID NO:18. In still other embodiments, said resistance-breaking ToBRFV isolate comprises an Asn to Lys substitution at position 82 of a ToBRFV movement protein. In specific embodiments, said resistance-breaking ToBRFV isolate comprises resistance-breaking ToBRFV isolate Vir353 (NCBI GenBank Accession OR760199).

In another aspect, a recombinant DNA segment from Solanum lycopersicum comprising a Tomato Brown Rugose Fruit Virus (ToBRFV) resistance allele from Solanum habrochaites that confers increased resistance to ToBRFV, wherein said recombinant DNA segment lacks an allele genetically linked thereto that confers an orange mature fruit color phenotype when present in a plant is provided. In some embodiments, said recombinant DNA segment comprises a sequence selected from the group consisting of SEQ ID NOs:12, 13, 14, and 35. In other embodiments, said recombinant DNA segment comprises an allele from Solanum lycopersicum at marker locus M12 (SEQ ID NO:12) and marker locus M14 (SEQ ID NO:14) and an allele from Solanum habrochaites at marker locus M13 (SEQ ID NO:13). In certain embodiments, said recombinant DNA segment further comprises an allele from Solanum lycopersicum at marker locus M17 (SEQ ID NO:35). In some embodiments, said recombinant DNA segment is further defined as comprised within a plant, plant part, plant cell, or seed.

In yet another aspect, methods are provided for producing a tomato plant with improved Tomato Brown Rugose Fruit Virus (ToBRFV) resistance, comprising introgressing into said plant at least one ToBRFV resistance allele within a recombinant chromosomal segment flanked in the genome of said plant by marker locus M12 (SEQ ID NO:12) and marker locus M14 (SEQ ID NO:14) on chromosome 8, wherein said introgressed ToBRFV resistance allele confers to said plant increased resistance to ToBRFV compared to a plant not comprising said allele, and wherein said recombinant chromosomal segment lacks an allele genetically linked thereto that confers an orange mature fruit color phenotype when present, and wherein said introgressing comprises marker-assisted selection. In some embodiments, introgressing further comprises backcrossing or assaying for said ToBRFV resistance. In other embodiments, said introgressing comprises: a) crossing a plant comprising said chromosomal segment with itself or with a tomato plant of a different genotype to produce at least a first progeny plant; and b) selecting a plant comprising said chromosomal segment. In some embodiments, marker-assisted selection comprises detecting at least a first marker locus genetically linked to said ToBRFV resistance allele selected from the group consisting of: marker locus M12 (SEQ ID NO:6), marker locus M14 (SEQ ID NO:14), marker locus M17 (SEQ ID NO:35), and marker locus M13 (SEQ ID NO:16). In other embodiments, said detecting comprises detecting an allele from Solanum lycopersicum at marker locus M12 (SEQ ID NO:12) and marker locus M14 (SEQ ID NO:14) and an allele from Solanum habrochaites at marker locus M13 (SEQ ID NO:13). In certain embodiments, the methods further comprise detecting an allele from Solanum lycopersicum at marker locus M17 (SEQ ID NO:35). Tomato plants obtainable by the methods disclosed herein are further provided.

### BRIEF DESCRIPTION OF THE DRAWINGS

FIG. 1: Shows leaf symptoms 14-days and 21-days post inoculation with a resistance-breaking Tomato Brown Rugose Fruit Virus isolate, Vir353 (NCBI GenBank Accession OR760199; Zisi et al., 2024), based on underlying genotype; and the Log(RQ) values for virus accumulation in such plants. The results shown for each underlying genotype represent twenty seedlings sown, planted in a randomized complete block design with four replications of five plants each, and inoculated 15 days after sowing. At 14 and 21 days post inoculation (DPI), virus symptoms were evaluated on leaflets. Symptom severity was assessed following the methodology of Gonzáles-Concha et al., with a rating scale with classes 1, 3, 5, 7, 9 where 1: no symptoms; 3: mild mosaic mottling hardly visible; 5: clear mosaic symptoms or mild mottling; 7: strong mosaic or clear mottling (leaf packed together); and 9: severe mosaic or strong mottling/misshaped leaf. Samples for virus titer analysis were taken at 21 DPI.

FIG. 2: Shows the level of resistance-breaking tomato brown rugose fruit virus accumulation in plants comprising QTL8B as compared to QTL8C using relative quantification (reference gene; Tip41). The p-value from the Wilcoxon rank sum test for the observed RQ is 0.0009973, which strongly suggests that the levels of virus accumulation are significantly different between plants comprising QTL8B and QTL8C.

### DETAILED DESCRIPTION OF THE INVENTION

Tomato brown rugose fruit virus (ToBRFV) is a devastating disease for major field and greenhouse vegetable crops grown all over the world, including tomatoes and peppers. ToBRFV was first isolated from greenhouse tomatoes in Jordan in April 2015, where disease incidence was close to 100% and brown rugose symptoms on fruits greatly affected fruit marketability. Around the same time, a similar Tobamovirus in tomatoes grown in net houses was reported in southern Israel. Molecular analysis confirmed high sequence identity between the Jordanian isolate (KT383474) and the Israeli isolate (GenBank KX619418). Symptoms of ToBRFV in tomato plants can vary, but typically include young leaves exhibiting mild to severe mosaic symptoms with dark green bulges, narrowness, and deformation. The peduncles and calyces often become necrotic and fail to produce fruit. Yellow blotches, brown or black spots, and rugose wrinkles appear on tomato fruits

ToBRFV spreads primarily through contaminated seeds and mechanical contact, such as standard horticultural practices. Since its initial discovery, the virus has spread rapidly and has been identified in 35 countries across four continents, including Asia, Europe, North America, and Africa. However, given the global seed trade and the virus's ability to transmit via seed, its spread is likely more extensive than reported. Furthermore, ToBRFV can break down genetic resistance to Tobamoviruses conferred by Tm-1, Tm-2, and Tm-22 resistance genes in tomato (Luria et al. (2017) PLoS One, 12, e01704). Pest management measures including crop rotation, eradication of infected plants, disinfection of seeds, and chemical treatment of contaminated greenhouses have achieved limited success.

Currently, ToBRFV is considered one of the most serious threats to tomato production in the world. As a plant RNA virus, ToBRFV has a high mutation rate and, in most cases, can exist in infected plants as a complex of different isolates (Harrison, 2002). Recent research has identified and characterized a single nucleotide ToBRFV mutant that is able to break resistance in new resistant tomato cultivars (Zisi et al., 2024). ToBRFV, and resistance-breaking (rb) ToBRFV isolates in particular, present a significant threat to tomato production.

Although some minimal to moderate resistance to ToBRFV has been observed with the Tm1 resistance gene under certain conditions and/or other known ToBRFV resistance QTLs on chromosome 8 and 11, developing compositions and methods that confer robust resistance to resistance-breaking ToBRFV isolates are of utmost importance. In particular, combinations of resistance loci that not only suppress virus multiplication and disease incidence but also reduce or eliminate transmission of resistance-breaking ToBRFV to susceptible tomato plants is of significant interest. As described herein, the present inventors identified a specific allele on chromosome 8 conferring resistance to resistance-breaking ToBRFV, as well as a combination of multiple resistance loci that confers further improvements in resistance to resistance-breaking ToBRFV isolates as compared to combinations known in the art.

The stacking of these multiple resistance loci can therefore provide much-needed long-term security against ToBRFV. The present disclosure represents a significant advance in that it provides, in one embodiment, a Solanum lycopersicum plant comprising: a Tm1 resistance gene on chromosome 2; a recombinant chromosomal segment on chromosome 11, wherein said chromosomal segment comprises a ToBRFV resistance allele; and a ToBRFV resistance gene on chromosome 8 encoding a ToBRFV resistance protein comprising a polypeptide sequence of SEQ ID NO:18 or 19; wherein the plant exhibits increased resistance to resistance-breaking ToBRFV isolates. Public entries carrying the Tm1 resistance gene include Master no 2 and NAK 83, and markers linked to Tm1 are described by Arens et al., (Theor Appl Genet 120(3):655-664, 2010). The recombinant chromosomal segment on chromosome 11 is located within a chromosomal segment flanked by marker locus M1 (SEQ ID NO:1) and marker locus M3 (SEQ ID NO:3) on chromosome 11. The ToBRFV resistance gene on chromosome 8 encodes a ToBRFV resistance protein comprising a polypeptide sequence of SEQ ID NO:18 or 19.

Surprisingly, it has been found that plants comprising the ToBRFV resistance gene on chromosome 8 encoding a ToBRFV resistance protein comprising a polypeptide sequence of SEQ ID NO:19 exhibit increased resistance to resistance-breaking ToBRFV, whereas plants comprising a ToBRFV resistance gene on chromosome 8 encoding a ToBRFV resistance protein comprising a polypeptide sequence of SEQ ID NO:18 do not exhibit such increased resistance. Therefore, in one aspect, provided herein are Solanum lycopersicum plants comprising: a Tm1 resistance gene on chromosome 2; a recombinant chromosomal segment on chromosome 11, wherein said chromosomal segment comprises a ToBRFV resistance allele; and a ToBRFV resistance gene on chromosome 8 encoding a ToBRFV resistance protein comprising a polypeptide sequence of SEQ ID NO:19; wherein the plant exhibits increased resistance to resistance-breaking ToBRFV isolates. In some embodiments, such Solanum lycopersicum plants comprising: a Tm1 resistance gene on chromosome 2; a recombinant chromosomal segment on chromosome 11, wherein said chromosomal segment comprises a ToBRFV resistance allele; and a ToBRFV resistance gene on chromosome 8 encoding a ToBRFV resistance protein comprising a polypeptide sequence of SEQ ID NO:19, may be referred to as exhibiting increased resistance to resistance-breaking ToBRFV isolates as compared to control plants comprising the Tm1 resistance gene, the recombinant chromosomal segment on chromosome 11, and a ToBRFV resistance gene on chromosome 8 encoding a ToBRFV resistance protein comprising a polypeptide sequence of SEQ ID NO:18.

The resistance profile and combination of resistance genes and QTLs are distinct from those known in the art, with significantly increased resistance when deployed together. For example, the tomato plants resistant to resistance-breaking ToBRFV isolates described herein may exhibit reduced ToBRFV-associated leaf symptoms, reduced ToBRFV-associated fruit symptoms, reduced ToBRFV transmissibility when infected, or reduced ToBRFV titer when exposed to resistance-breaking ToBRFV isolates, as compared to tomato plants known in the art. In addition, markers for the loci are provided, allowing at least one locus, at least two loci, or three loci, to be accurately introgressed and tracked during development of new varieties. As such, the invention permits introgression of the disease resistance loci into potentially any desired tomato genotype.

In certain embodiments, Solanum lycopersicum plants are provided herein comprising a Tm1 resistance gene on chromosome 2; a recombinant chromosomal segment on chromosome 11, wherein said chromosomal segment comprises a ToBRFV resistance allele; and a ToBRFV resistance gene on chromosome 8 encoding a ToBRFV resistance protein comprising a polypeptide sequence of SEQ ID NO:18 or 19; wherein the plant exhibits increased resistance to resistance-breaking ToBRFV isolates. Such plants, in some embodiments, exhibit reduced ToBRFV-associated leaf symptoms when exposed to resistance-breaking ToBRFV isolates, reduced ToBRFV transmissibility when infected with resistance-breaking ToBRFV isolates, or reduced ToBRFV titer when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19.

In certain embodiments, SNP markers M1, M2, and M3, can be used to select for the rbToBRFV resistance trait on chromosome 11. Additional markers that can be used to select for the ToBRFV resistance trait are M4, a SNP marker with a [T/G] change at 8,891,489 bp on chromosome 11 of the public tomato genome map version SL2.50; M5, a SNP marker with a [C/T] change at 9,355,794 bp on chromosome 11 of the public tomato genome map version SL2.50; M6, a SNP marker with a [A/T] change at 9,401,319 bp on chromosome 11 of the public tomato genome map version SL2.50; M7, a SNP marker with a [G/T] change at 9,406,414 bp on chromosome 11 of the public tomato genome map version SL2.50; M8, a SNP marker with a [A/T] change at 9,421,426 bp on chromosome 11 of the public tomato genome map version SL2.50; M9, a SNP marker with a [T/C] change at 9,470,789 bp on chromosome 11 of the public tomato genome map version SL2.50; M10, a SNP marker with a [A/G] change at 9,756,371 bp on chromosome 11 of the public tomato genome map version SL2.50. Such markers (M1-M10) are disclosed and described in U.S. Pat. No. 11,162,110, which is incorporated herein by reference in its entirety. Furthermore, M11 described herein, a SNP marker with a [A/G] change at 9,521,767 bp on chromosome 11 of the public tomato genome map version SL2.50 can also be used to select for the ToBRFV resistance trait.

In other embodiments, Solanum lycopersicum plants are provided herein comprising a Tm1 resistance gene on chromosome 2; a ToBRFV resistance gene on chromosome 8 encoding a ToBRFV resistance protein comprising a polypeptide sequence of SEQ ID NO:19; and recombinant chromosomal region on chromosome 11 comprising a ToBRFV resistance allele, wherein said resistance allele is flanked in the genome by marker locus M20 (also known as TO-0122252; SEQ ID NO:20) and marker locus M31 (also known as TO-0162427; SEQ ID NO:31) in said plant; and wherein the plant exhibits increased resistance to resistance-breaking ToBRFV isolates. Additional markers that can be used to select for the recombinant chromosomal region on chromosome 11 comprising a ToBRFV resistance allele, wherein said resistance allele is flanked in the genome by marker locus M20 (SEQ ID NO:20) and marker locus M31 (SEQ ID NO:31) include M21-M30 (also known as TO-0144317, TO-0142270, TO-0142294, TO-0142303, TO-0142306, TO-0182276, TO-0181040, TO-0123057, TO-0125528, and TO-0162432, respectively; SEQ ID NOs:21-30). Said marker loci as well as plants comprising said quantitative trait locus on chromosome 11 flanked in the genome by marker locus M20 (also known as TO-0122252; SEQ ID NO:20) and marker locus M31 (also known as TO-0162427; SEQ ID NO:31) are described in U.S. Pat. No. 11,889,812 (referred to as QTL3).

In some embodiments, plants comprising a Tm1 resistance gene on chromosome 2; a ToBRFV resistance gene on chromosome 8 encoding a ToBRFV resistance protein comprising a polypeptide sequence of SEQ ID NO:19; and a recombinant chromosomal region on chromosome 11 comprising a ToBRFV resistance allele, wherein said resistance allele is flanked in the genome by marker locus M20 (also known as TO-0122252; SEQ ID NO:20) and marker locus M31 (also known as TO-0162427; SEQ ID NO:31) in said plant, and wherein the plant exhibit reduced ToBRFV-associated leaf symptoms when exposed to resistance-breaking ToBRFV isolates, reduced ToBRFV transmissibility when infected with resistance-breaking ToBRFV isolates, or reduced ToBRFV titer when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19.

In some embodiments, the ToBRFV resistance protein may be described as comprising lysine to glutamic acid mutation (K223E) at position 223 of SEQ ID NO:18. In other embodiments, the ToBRFV resistance gene on chromosome 8 may be described as comprising an A to G nucleotide substitution at position 667 of SEQ ID NO:18. In certain embodiments, the ToBRFV resistance protein may be described as comprising a glutamate at position 223 and otherwise comprising at least 95%, 96%, 97% 98%, or 99% sequence identity to SEQ ID NO:18 or 19. In specific embodiments, the ToBRFV resistance gene on chromosome 8 may be described as comprising a guanine at position 667 and otherwise comprising at least 90%, 95%, 96%, 97% 98%, or 99% sequence identity to SEQ ID NO:18 or 19.

The tomato reference genome is publicly available, for example, at www.solgenomics.net, and one skilled in the art would understand that the marker sequences provided for the first time in the instant application could be located on any version (or later version) of the public genome.

In certain embodiments, the invention provides methods of producing or selecting a tomato plant exhibiting resistance to resistance-breaking ToBRFV isolates by a) crossing a tomato plant provided herein with itself or with a second tomato plant of a different genotype to produce one or more progeny plants; and b) selecting a progeny plant comprising the Tm1 resistance gene on chromosome 2, the recombinant chromosomal segment on chromosome 11, and the ToBRFV resistance gene on chromosome 8. In some embodiments, methods of the invention comprise selecting a progeny plant by detecting nucleic acids comprising marker locus marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), M12 (SEQ ID NO:12), M13 (SEQ ID NO:13), M17 (SEQ ID NO:35), or M14 (SEQ ID NO:14). In particular embodiments, selecting a tomato plant exhibiting resistance to resistance-breaking ToBRFV comprises molecular genetic techniques. For example, those of ordinary skill in the art viewing the present disclosure may use technical methods to select a tomato plant exhibiting resistance to resistance-breaking ToBRFV by screening one or more plants with at least one nucleic acid marker to detect a polymorphism genetically linked to ToBRFV resistance.

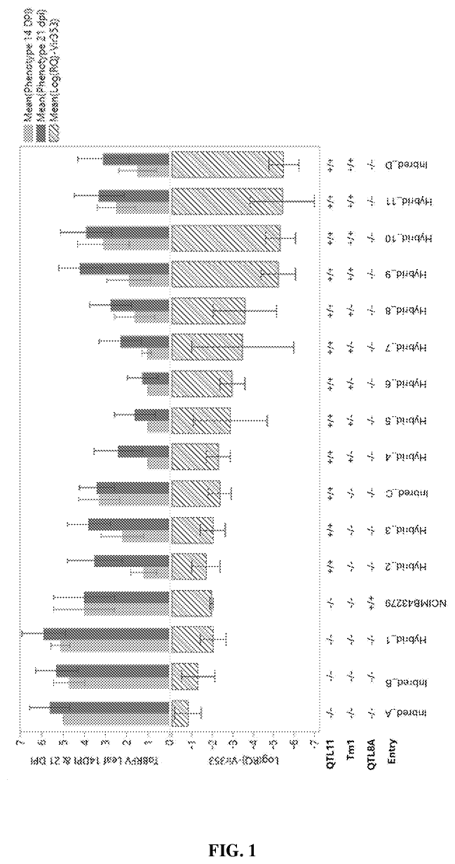

As described herein, tomato varieties comprising a recombinant chromosomal segment on chromosome 11, wherein said chromosomal segment comprises a ToBRFV resistance allele and the Tm1 resistance gene (in homozygous or heterozygous form) show fewer symptoms as compared to susceptible controls lacking the recombinant chromosomal segment on chromosome 11 and the Tm1 resistance gene at 14 and 21 days post inoculation (DPI), the reduction in symptoms being more pronounced at 14 DPI. Additionally, plants comprising the recombinant chromosomal segment on chromosome 11 and the Tm1 resistance gene in homozygous form show significantly reduced virus multiplication as compared to susceptible controls lacking the recombinant chromosomal segment on chromosome 11 and the Tm1 resistance gene. A less pronounced trend regarding reduced virus multiplication is observed in plants comprising Tm1 in heterozygous form as compared to susceptible controls lacking the recombinant chromosomal segment on chromosome 11 and the Tm1 resistance gene (See FIG. 1). Therefore, in another aspect, provided herein is a Solanum lycopersicum plant comprising a Tm1 resistance gene on chromosome 2; and a recombinant chromosomal segment on chromosome 11, wherein said chromosomal segment comprises a ToBRFV resistance allele; and wherein the plant exhibits increased resistance to resistance-breaking ToBRFV isolates. In certain embodiments, said plant is homozygous for the Tm1 resistance gene and the ToBRFV resistance allele. In further embodiments, said plant exhibits reduced ToBRFV-associated leaf symptoms when exposed to resistance-breaking ToBRFV isolates as compared to a control plant lacking the Tm1 resistance gene, and the ToBRFV resistance allele.

Also provided herein, is a method for controlling or preventing ToBRFV symptoms during crop production, wherein the ToBRFV symptoms are caused by resistance-breaking ToBRFV isolates, comprising obtaining a population of Solanum lycopersicum plants comprising a Tm1 resistance gene on chromosome 2; and a recombinant chromosomal segment on chromosome 11, wherein said chromosomal segment comprises a ToBRFV resistance allele; growing the Solanum lycopersicum plants; and harvesting fruit of the Solanum lycopersicum plants. Such Solanum lycopersicum plant comprising a Tm1 resistance gene on chromosome 2; and a recombinant chromosomal segment on chromosome 11, wherein said chromosomal segment comprises a ToBRFV resistance allele may also be used for improving tomato crop yield in an area comprising resistance-breaking ToBRFV isolates.

Because genetically diverse plant lines can be difficult to cross, the introgression of ToBRFV resistance loci and/or alleles into cultivated lines using conventional breeding methods could require prohibitively large segregating populations for progeny screens with an uncertain outcome. Marker-assisted selection (MAS) is therefore essential for the effective introgression of loci that confer resistance to resistance-breaking ToBRFV isolates into elite cultivars. The present disclosure enables effective MAS by providing improved and validated markers for detecting genotypes associated with resistance to resistance-breaking ToBRFV isolates without the need to grow large populations of plants to maturity in order to observe the phenotype.

TABLE 1
List of markers and favorable alleles at each marker for tracking resistance QTLs.

| | | | | | SNP | | | |
|---|---|---|---|---|---|---|---|---|
| | | | Public | | position | | | Marker |
| | | Genetic | position | Marker | in | | | sequence |
| | | Position | SNP** | size | marker | SNP | Favorable | (SEQ ID |
| Marker name | Chr. | (cM)* | (bp) | (bp) | (bp) | change | allele | NO) |
| M1 | 11 | 53.33 | 8,894,829 | 1312 | 529 | [A/C] | A | 1 |
| M2 | 11 | 54.85 | 9,591,834 | 199 | 99 | [G/A] | A | 2 |
| M3 | 11 | 55.40 | 9,826,973 | 1968 | 787 | [A/T] | A | 3 |
| M4 | 11 | 53.32 | 8,891,489 | 101 | 51 | [T/G] | G | 4 |
| M5 | 11 | 54.26 | 9,355,794 | 201 | 101 | [C/T] | T | 5 |
| M6 | 11 | 54.36 | 9,401,319 | 201 | 101 | [A/T] | T | 6 |
| M7 | 11 | 54.37 | 9,406,414 | 201 | 101 | [G/T] | T | 7 |
| M8 | 11 | 55.40 | 9,421,426 | 201 | 101 | [A/T] | T | 8 |
| M9 | 11 | 54.53 | 9,470,789 | 187 | 101 | [T/C] | C | 9 |
| M10 | 11 | 55.24 | 9,756,371 | 184 | 101 | [A/G] | G | 10 |
| M11 | 11 | 54.66 | 9,521,767 | 1001 | 587 | [A/G] | G | 11 |
| M15 | 8 | 57.68 | 59,306,115 | 301 | 151 | [T/A] | T | 33 |
| M16 | 8 | 60.67 | 59,673,267 | 301 | 151 | [A/G] | A | 34 |
| M12 | 8 | 61.01 | 59,715,652 | 301 | 151 | [C/G] | C | 12 |
| M13 | 8 | 61.4 | 59,762,442 | 301 | 151 | [C/T] | T | 13 |
| M17 | 8 | 62.01 | 59,836,842 | 301 | 151 | [C/A] | C | 35 |
| M14 | 8 | 63.11 | 59,968,063 | 301 | 151 | [C/T] | T | 14 |
| M18 | 8 | 63.68 | 60,038,311 | 301 | 151 | [C/T] | T | 36 |
| M20 (TO-0122252) | 11 | 50.80 | 8,090,364 | 101 | 51 | [A/T] | T | 20 |
| M21 (TO-0144317) | 11 | 51.67 | 8,334,467 | 101 | 51 | [T/C] | C | 21 |
| M22 (TO-0142270) | 11 | 52.60 | 8,633,469 | 101 | 51 | [C/T] | T | 22 |
| M23 (TO-0142294) | 11 | 52.97 | 8,764,030 | 101 | 51 | [A/G] | G | 23 |
| M24 (TO-0142303) | 11 | 53.35 | 8,903,092 | 101 | 51 | [C/A] | A | 24 |
| M25 (TO-0142306) | 11 | 54.19 | 9,318,832 | 101 | 51 | [G/A] | A | 25 |
| M26 (TO-0182276) | 11 | 54.73 | 9,548,029 | 160 | 59 | [A/G] | G | 26 |
| M27 (TO-0181040) | 11 | 55.33 | 9,797,143 | 101 | 51 | [A/G] | G | 27 |
| M28 (TO-0123057) | 11 | 55.33 | 9,825,111 | 101 | 51 | [T/G] | G | 28 |
| M29 (TO-0125528) | 11 | 55.42 | 9,837,711 | 121 | 61 | [G/A] | A | 29 |
| M30 (TO-0162432) | 11 | 55.75 | 10,015,478 | 101 | 51 | [T/C] | C | 30 |
| M31 (TO-0162427) | 11 | 55.79 | 10,018,811 | 101 | 51 | [C/T] | T | 31 |
| M32 | 2 | 9.10 | 34,281,808 | 117 | 49 | [G/A] | G | 32 |

*inferred for the SL2.50 physical assembly from genetic map data
**with reference to tomato genome physical assembly version SL2.50

### I. Genomic Region, QTL, Polymorphic Nucleic Acids, and Alleles Associated with ToBRFV Resistance

The invention provides novel combinations of loci associated with, and conferring, resistance to resistance-breaking ToBRFV isolates in tomato, together with polymorphic nucleic acids and linked markers for tracking the introgressions during plant breeding. Public entries carrying the Tm1 resistance gene include Master no 2 and NAK 83, which may be used as a source for the Tm1 resistance gene. Markers linked to Tm1 are described by Arens et al. (Theor Appl Genet 120(3):655-664, 2010). The seeds deposited under NCMA Accession No. 202103011 may be used as a source for the recombinant chromosomal segment on chromosome 11, the introgression of which may be tracked using marker locus M1 (SEQ ID NO:1), marker locus M2 (SEQ ID NO:2), marker locus M3 (SEQ ID NO:3), or a combination of any thereof. Public entries carrying a ToBRFV resistance gene on chromosome 8 encoding a ToBRFV resistance protein comprising a polypeptide sequence of SEQ ID NO:19 include LA2812, available from the C. M. Rick Tomato Genetics Resource Center (tgrc-mvc.plantsciences.ucdavis.edu/Accession/detail/LA2812). Additionally, seeds comprising the Tm1 resistance gene, the chromosomal segment on chromosome 11, and the ToBRFV resistance gene QTL8B (SEQ ID NO:16) on chromosome 8 described herein, are deposited under NCMA Accession No. 202504007, which is an F₁ hybrid produced from a cross of an inbred parent homozygous for the Tm1 and the chromosomal segment on chromosome 11 and plants of LA2812 comprising the ToBRFV resistance gene QTL8B (SEQ ID NO:16) on chromosome 8. The seeds deposited under NCMA Accession No. 202504007 may be used as a source for the Tm1 resistance gene, the chromosomal segment on chromosome 11, the ToBRFV resistance gene QTL8B (SEQ ID NO:16) on chromosome 8, or any combination thereof, the introgression of which may be tracked using marker loci described herein.

Using the improved genetic markers and assays of the invention, the present inventors were able to successfully identify novel combinations of loci that confer to a tomato plant resistance to resistance-breaking ToBRFV isolates.

### II. Introgression of a Genomic Locus Associated with Resistance to ToBRFV in Tomato

Marker-assisted introgression involves the transfer of a chromosomal region defined by one or more markers from a first genetic background to a second. Offspring of a cross that contain the introgressed genomic region can be identified by the combination of markers characteristic of the desired introgressed genomic region from a first genetic background and both linked and unlinked markers characteristic of the second genetic background.

Provided herein are accurate markers for identifying and tracking introgression of one or more of the genomic regions disclosed herein from a ToBRFV resistant plant into a cultivated line. The invention further provides markers for identifying and tracking the novel combination of introgressions disclosed herein during plant breeding, including the markers set forth in Table 1.

Markers within or linked to any of the genomic intervals of the present disclosure may be useful in a variety of breeding efforts that include introgression of genomic regions associated with resistance to resistance-breaking ToBRFV isolates into a desired genetic background. For example, a marker within 40 cM, 20 cM, 15 CM, 10 cM, 5 CM, 2 cM, or 1 cM of a marker associated with ToBRFV resistance described herein can be used for marker-assisted introgression of genomic regions associated with a ToBRFV resistant phenotype.

Markers that are linked and either immediately adjacent or adjacent to the identified ToBRFV resistance loci that permit introgression of the loci in the absence of extraneous linked DNA from the source germplasm containing the loci are provided herewith. Those of skill in the art will appreciate that when seeking to introgress a smaller genomic region comprising a QTL or resistance loci associated with resistance to ToBRFV described herein, that any of the telomere proximal or centromere proximal markers that are immediately adjacent to a larger genomic region comprising the QTL can be used to introgress that smaller genomic region.

Tomato plants or germplasm comprising at least one introgressed region (or a combination of introgressed regions) that is associated with resistance to resistance-breaking ToBRFV isolates wherein at least 10%, 25%, 50%, 75%, 90%, or 99% of the remaining genomic sequences carry markers characteristic of plant or germplasm that otherwise or ordinarily comprise a genomic region associated with another phenotype, are thus provided in specific embodiments. Furthermore, tomato plants comprising at least one introgressed region (or a combination of introgressed regions) where closely linked regions adjacent and/or immediately adjacent to the genomic regions, QTL, and markers provided herewith that comprise genomic sequences carrying markers characteristic of tomato plants or germplasm that otherwise or ordinarily comprise a genomic region associated with the phenotype are also provided.

### III. Development of Tomato Plants with Resistance to ToBRFV

For most breeding objectives, commercial breeders may work within germplasm that is often referred to as “cultivated” or “elite.” This germplasm is easier to use in plant breeding because it generally performs well when evaluated for horticultural performance. The performance advantage a cultivated variety provides is sometimes offset by a lack of allelic diversity. Breeders generally accept this tradeoff because progress is faster when working with cultivated material than when breeding with genetically diverse sources.

In contrast, when cultivated germplasm is crossed with non-cultivated germplasm, a breeder can gain access to novel alleles from the non-cultivated type. However, this approach presents significant difficulties due to fertility problems associated with crosses between diverse lines, and negative linkage drag from the non-cultivated parent. In tomato plants, non-cultivated types can provide alleles associated with disease resistance. However, these non-cultivated types may have poor horticultural qualities.

The process of introgressing desirable resistance genes from non-cultivated lines into elite cultivated lines while avoiding problems with genetically linked deleterious loci or low heritability is a long and often arduous process. In deploying loci derived from wild relatives it is often desirable to introduce a minimal or truncated introgression that provides the desired trait but lacks detrimental effects. To aid introgression reliable marker assays are preferable to phenotypic screens. Success is furthered by simplifying genetics for key attributes to allow focus on genetic gain for quantitative traits such as ToBRFV resistance. Moreover, the process of introgressing genomic regions from non-cultivated lines can be greatly facilitated by the availability of accurate markers for MAS.

One of skill in the art would therefore understand that the loci, polymorphisms, and markers provided by the invention allow the tracking and introduction of any of the genomic regions identified herein into any genetic background. In addition, the genomic regions associated with ToBRFV resistance disclosed herein can be introgressed from one genotype to another and tracked using MAS. Thus, the inventors' discovery of accurate markers associated with ToBRFV resistance will facilitate the development of tomato plants having beneficial phenotypes. For example, seed can be genotyped using the markers of the present disclosure to select for plants comprising desired genomic regions associated with resistance to resistance-breaking ToBRFV isolates. Moreover, MAS allows identification of plants homozygous or heterozygous for a desired introgression.

Inter-species crosses can also result in suppressed recombination and plants with low fertility or fecundity. For example, suppressed recombination has been observed for the tomato nematode resistance gene Mi, the Mla and Mlg genes in barley, the Yr17 and Lr20 genes in wheat, the Run1 gene in grapevine, and the Rma gene in peanut. Meiotic recombination is essential for classical breeding because it enables the transfer of favorable loci across genetic backgrounds, the removal of deleterious genomic fragments, and pyramiding traits that are genetically tightly linked. Therefore, suppressed recombination forces breeders to enlarge segregating populations for progeny screens in order to arrive at the desired genetic combination.

Phenotypic evaluation of large populations is time-consuming, resource-intensive and not reproducible in every environment. Marker-assisted selection offers a feasible alternative. Molecular assays designed to detect unique polymorphisms, such as SNPs, are versatile. However, they may fail to discriminate loci within and among tomato species in a single assay. Structural rearrangements of chromosomes such as deletions impair hybridization and extension of synthetically labeled oligonucleotides. In the case of duplication events, multiple copies are amplified in a single reaction without distinction. The development and validation of accurate and highly predictive markers are therefore essential for successful MAS breeding programs.

### IV. Marker-Assisted Breeding and Genetic Engineering Techniques

Genetic markers that can be used in the practice of the present invention include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art. Marker discovery and development in crop plants provides the initial framework for applications to marker-assisted breeding activities (U.S. Patent Pub. Nos. 2005/0204780; 2005/0216545; 2005/0218305; and 2006/00504538). The resulting “genetic map” is the representation of the relative position of characterized loci (polymorphic nucleic acid markers or any other locus for which loci can be identified) to each other.

Polymorphisms comprising as little as a single nucleotide change can be assayed in a number of ways. For example, detection can be made by electrophoretic techniques including a single strand conformational polymorphism (Orita et al., Genomics 8(2):271-278, 1989), denaturing gradient gel electrophoresis (Myers, EP0273085), or cleavage fragment length polymorphisms (Life Technologies, Inc., Gaithersburg, MD), but the widespread availability of DNA sequencing often makes it easier to simply sequence amplified products directly. Once the polymorphic sequence difference is known, rapid assays can be designed for progeny testing, typically involving some version of PCR amplification of specific loci (PASA; Sommer et al., Biotechniques 12(1):82-87, 1992), or PCR amplification of multiple specific loci (PAMSA; Dutton and Sommer, Biotechniques 11(6):700-7002, 1991).

Polymorphic markers serve as useful tools for assaying plants for determining the degree of identity of lines or varieties (U.S. Pat. No. 6,207,367). These markers form the basis for determining associations with phenotypes and can be used to drive genetic gain. In certain embodiments of methods of the invention, polymorphic nucleic acids can be used to detect in a tomato plant a genotype associated with resistance to resistance-breaking ToBRFV isolates, identify a tomato plant with a genotype associated with resistance to resistance-breaking ToBRFV isolates, and to select a tomato plant with a genotype associated with resistance to resistance-breaking ToBRFV isolates. In certain embodiments of methods of the invention, polymorphic nucleic acids can be used to produce a tomato plant that comprises in its genome at least one introgressed locus associated with resistance to resistance-breaking ToBRFV isolates. In certain embodiments of the invention, polymorphic nucleic acids can be used to breed progeny tomato plants comprising a locus or loci associated with resistance to resistance-breaking ToBRFV isolates.

Genetic markers may include “dominant” or “codominant” markers. “Codominant” markers reveal the presence of two or more loci (two per diploid individual). “Dominant” markers reveal the presence of only a single locus. Markers are preferably inherited in codominant fashion so that the presence of both loci at a diploid locus, or multiple loci in triploid or tetraploid loci, are readily detectable, and they are free of environmental variation, i.e., their heritability is 1. A marker genotype typically comprises two marker loci at each locus in a diploid organism. The marker allelic composition of each locus can be either homozygous or heterozygous. Homozygosity is a condition where both loci at a locus are characterized by the same nucleotide sequence. Heterozygosity refers to a condition where the two loci at a locus are different.

Nucleic acid-based analyses for determining the presence or absence of the genetic polymorphism (i.e. for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, loci, or genomic regions that comprise or are linked to a genetic marker that is linked to or associated with ToBRFV resistance in tomato plants.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al., Cold Spring Harbor Symp. Quant. Biol. 51:263-273, 1986; European Patent No. 50,424; European Patent No. 84,796; European Patent No. 258,017; European Patent No. 237,362; European Patent No. 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981; and 7,250,252 all of which are incorporated herein by reference in their entirety. However, the compositions and methods provided herein can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to locus-specific oligonucleotide (ASO) probes as disclosed in U.S. Pat. Nos. 5,468,613 and 5,217,863. U.S. Pat. No. 5,468,613 discloses locus specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523, 2003; Cui et al., Bioinformatics 21:3852-3858, 2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, a locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays.

Various genetic engineering technologies have been developed and may be used by those of skill in the art to introduce traits in plants. In certain aspects of the claimed invention, traits are introduced into tomato plants via altering or introducing a single genetic locus or transgene into the genome of a variety or progenitor thereof. Methods of genetic engineering to modify, delete, or insert genes and polynucleotides into the genomic DNA of plants are well-known in the art.

In specific embodiments of the invention, improved tomato lines can be created through the site-specific modification of a plant genome. Methods of genetic engineering include, for example, utilizing sequence-specific nucleases such as zinc-finger nucleases (see, for example, U.S. Patent Pub. No. 2011/0203012); engineered or native meganucleases; TALE-endonucleases (see, for example, U.S. Pat. Nos. 8,586,363 and 9,181,535); and RNA-guided endonucleases, such as those of the CRISPR/Cas systems (see, for example, U.S. Pat. Nos. 8,697,359 and 8,771,945 and U.S. Patent Pub. No. 2014/0068797). One embodiment of the invention thus relates to utilizing a nuclease or any associated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a plant genome (see, for example, Sauer et al., Plant Physiol. 170(4):1917-1928, 2016).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick. The presence of homology arms in the DNA to be integrated may promote the adoption and targeting of the insertion sequence into the plant genome during the repair process through homologous recombination or non-homologous end joining (NHEJ).

In another embodiment of the invention, genetic transformation may be used to insert a selected transgene into a plant of the invention or may, alternatively, be used for the preparation of transgenes which can be introduced by backcrossing. Methods for the transformation of plants that are well-known to those of skill in the art and applicable to many crop species include, but are not limited to, electroporation, microprojectile bombardment, Agrobacterium-mediated transformation, and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. Microprojectile bombardment techniques are widely applicable and may be used to transform virtually any plant species.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., Nat. Biotechnol. 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., Nat. Biotechnol. 3:629-635, 1985; U.S. Pat. No. 5,563,055).

Transformation of plant protoplasts also can be achieved using methods based on calcium phosphate precipitation, polyethylene glycol treatment, electroporation, and combinations of these treatments (see, for example, Potrykus et al., Mol. Gen. Genet. 199:183-188, 1985; Omirulleh et al., Plant Mol. Biol. 21(3):415-428, 1993; Fromm et al., Nature 312:791-793, 1986; Uchimiya et al., Mol. Gen. Genet. 204:204, 1986; Marcotte et al., Nature 335:454, 1988). Transformation of plants and expression of foreign genetic elements is exemplified in Choi et al. (Plant Cell Rep. 13:344-348, 1994), and Ellul et al. (Theor. Appl. Genet. 107:462-469, 2003).

### V. Definitions

The following definitions are provided to better define the present invention and to guide those of ordinary skill in the art in the practice of the present invention. Unless otherwise noted, terms are to be understood according to conventional usage by those of ordinary skill in the relevant art.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

As used herein, the term “plant” includes the seed (from which the plant can be grown), the whole plant or any plant parts, such as plant organs (e.g., harvested or non-harvested leaves, fruit, etc.), plant cells, plant protoplasts, plant cell- or tissue cultures from which whole plants can be regenerated, propagating or non-propagating plant cells, plants cells which are not in tissue culture (but which are, for example, in vivo in a plant or plant part), plant callus, plant cell clumps, plant transplants, seedlings, plant cells that are intact in plants, plant clones or micro-propagations, or parts of plants (e.g., harvested tissues or organs), such as plant cuttings, vegetative propagations, embryos, pollen, ovules, flowers, leaves, heads, seeds (produced on the plant after self-fertilization or cross-fertilization), clonally propagated plants, roots, stems, stalks, root tips, grafts, parts of any of these and the like, or derivatives thereof, preferably having the same genetic make-up (or very similar genetic make-up) as the plant from which it is obtained. Also any developmental stage is included, such as seedlings, cuttings prior or after rooting, mature and/or immature plants or mature and/or immature leaves. When “seeds of a plant” are referred to, these either refer to seeds from which the plant can be grown or to seeds produced on the plant, after self-fertilization or cross-fertilization.

As used herein, the term “population” means a genetically heterogeneous collection of plants that share a common parental derivation.

As used herein, the term “plant line” is, for example, a breeding line which can be used to develop one or more varieties. “Inbred line” or “inbred parent” is a line which has been developed by selfing for several generations and which can be used as a parent to produce an F₁ hybrid variety. “Hybrid” refers to the seeds harvested from crossing one plant line or variety with another plant line or variety, and the plants or plant parts grown from said seeds.

As used herein, the term “F₁ hybrid” plant (or F₁ hybrid seed) is the generation obtained from crossing two non-isogenic inbred parent lines. Thus, F₁ hybrid seeds are seeds from which F₁ hybrid plants grow.

As used herein, the term “interspecific hybrid” refers to a hybrid produced from crossing a plant of one species, e.g., Solanum lycopersicum, with a plant of another species, e.g., Solanum pimpinellifolium.

As used herein, the terms “progeny,” “progenies,” or “descendants” as used herein, refer to offspring, or the first and all further descendants derived from (or obtainable from) a plant. Progeny may be derived by regeneration of cell culture or tissue culture, or parts of a plant, or selfing of a plant, or by producing seeds of a plant. In further embodiments, progeny may also encompass tomato plants derived from crossing of at least one tomato plant with another tomato plant of the same or another variety or (breeding) line, and/or backcrossing, and/or inserting of a locus into a plant and/or mutation. A progeny is, e.g., a first generation progeny, i.e., the progeny is directly derived from, obtained from, obtainable from or derivable from the parent plant by, e.g., traditional breeding methods (selfing and/or crossing) or regeneration. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above. Also double haploid plants are progeny.

As used herein, the term “tissue culture” or “cell culture” refers to an in vitro composition comprising isolated cells of the same or a different type or a collection of such cells organized into plant tissue. Tissue cultures and cell cultures of tomato, and regeneration of tomato plants therefrom, is well known in the art and widely published.

As used herein, the term “regeneration” refers to the development of a plant from in vitro cell culture or tissue culture or vegetative propagation.

As used herein, the term “vegetative propagation,” “vegetative reproduction,” and “clonal propagation” are used interchangeably herein and refer to the method of taking part of a plant and allowing that plant part to form at least roots where plant part is, e.g., defined as or derived from (e.g., by cutting off) leaf, pollen, embryo, cotyledon, hypocotyl, cells, protoplasts, meristematic cell, root, root tip, pistil, anther, flower, shoot tip, shoot, stem, fruit, and petiole. When a whole plant is regenerated by vegetative propagation, it is also referred to as a “vegetative propagation” or a “vegetatively propagated plant.”

As used herein, the term “harvested plant material” refers to plant parts (e.g., leaves detached from the whole plant) which have been collected for further storage and/or further use. As used herein, the term “harvested seeds” refers to seeds harvested from a line or variety, e.g., produced after self-fertilization or cross-fertilization and collected. As used herein, the term “harvested leaves” refers to tomato leaves, i.e., the plant without the root system, for example substantially all (harvested) leaves.

As used herein, the terms “variety” and “cultivar” mean a group of similar plants that by their genetic pedigrees and performance can be identified from other varieties within the same species.

As used herein, “elite” or “cultivated” variety means any variety that has resulted from breeding and selection for superior agronomic performance. An “elite plant” refers to a plant belonging to an elite variety. Numerous elite varieties are available and known to those of skill in the art of tomato breeding. An “elite population” is an assortment of elite individuals or varieties that can be used to represent the state of the art in terms of agronomically superior genotypes of a given crop species, such as tomato. Similarly, an “elite germplasm” or elite strain of germplasm is an agronomically superior germplasm. An “elite tomato” or “cultivated tomato” cultivar/variety refers herein to plants of the species Solanum lycopersicum (or seeds from which the plants can be grown), and parts of such plants, bred by humans for food and having good agronomic characteristics. This includes any cultivated tomato, such as breeding lines (e.g. backcross lines, inbred lines), cultivars, and varieties (open-pollinated or hybrids). Wild tomato (i.e. not cultivated tomato) such as Solanum pimpinellifolium or Solanum habrochaites, or wild relatives of tomato are not encompassed by this definition.

As used herein, an “allele” refers to one of two or more alternative forms of a genomic sequence at a given locus on a chromosome. All alleles at a specific locus relate to one trait or characteristic. In a diploid cell of an organism, alleles of a given gene are located at a specific location, or locus on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

As used herein, “Tomato Brown Rugose Fruit Virus (ToBRFV),” “TBRFV,” and “ToBRFV” refers to a disease of plants caused by a virus of the Virgaviridae family.

As used herein, “breaking isolate” and “resistance-breaking isolate” refer to newly occurring strains of ToBRFV that can overcome resistance conferred by known ToBRFV resistance alleles.

As used herein, the term “locus” or “loci” refers to a specific place or places or a site on a chromosome where, for example, a gene or genetic marker is found. A “quantitative trait locus (QTL)” is a chromosomal location that encodes for at least a first allele that affects the expressivity of a phenotype.

As used herein, an “introgression fragment” or “introgression segment” or “introgression region” refers to a chromosome fragment (or chromosome part or region) which has been introduced into another plant of the same or related species by crossing or traditional breeding techniques, such as backcrossing, i.e., the introgressed fragment is the result of breeding methods referred to by the verb “to introgress” (such as backcrossing). In tomato, wild tomato, or wild relatives of tomato are used to introgress fragments of the wild genome into the genome of cultivated tomato. Such a tomato plant thus has a “genome of Solanum lycopersicum” but comprises in the genome a fragment of a wild tomato or tomato relative, i.e., an introgression fragment of a donor plant. It is understood that the term “introgression fragment” never includes a whole chromosome, but only a part of a chromosome.

As used herein, the terms “distal” and “proximal” describe the position of a chromosomal segment or an introgression segment in relation to a specific reference point on a whole chromosome, i.e. “distal” means that the interval or the segment is localized on the side of the reference point distant from the chromosome centromere, and “proximal” means that the interval or the segment is localized on the side of the reference point close to the chromosome centromere.

A genetic element, a locus, an introgression fragment or a gene or allele conferring a trait (such as resistance to resistance-breaking ToBRFV isolates) is said to be “obtainable from” or can be “obtained from” or “derivable from” or can be “derived from” or “as present in” or “as found in” a plant or seed if it can be transferred from the plant or seed in which it is present into another plant or seed in which it is not present (such as a line or variety) using traditional breeding techniques without resulting in a phenotypic change of the recipient plant apart from the addition of the trait conferred by the genetic element, locus, introgression fragment, gene, or allele. The terms are used interchangeably and the genetic element, locus, introgression fragment, gene, or allele can thus be transferred into any other genetic background lacking the trait. Not only seeds deposited and comprising the genetic element, locus, introgression fragment, gene, or allele can be used, but also progeny/descendants from such seeds which have been selected to retain the genetic element, locus, introgression fragment, gene, or allele, can be used and are encompassed herein, such as commercial varieties developed from the deposited seeds or from descendants thereof. Whether a plant comprises the same genetic element, locus, introgression fragment, gene, or allele as obtainable from the deposited seeds can be determined by the skilled person using one or more techniques known in the art, such as phenotypic assays, whole genome sequencing, molecular marker analysis, trait mapping, chromosome painting, allelism tests, and the like.

As used herein, a “marker” refers to detectable characteristic that can be used to discriminate between organisms. Examples of such characteristics include, but are not limited to, genetic markers, biochemical markers, metabolites, morphological characteristics, and agronomic characteristics.

As used herein, a “molecular marker” is a piece of DNA associated with a certain genomic or chromosomal location or single nucleotide polymorphism (SNP), which is found on the chromosome close to the gene of interest. Molecular markers can be used to identify a particular sequence of DNA, or a certain location in a genome or on a chromosome, or to identify an introgression fragment. When reference is made herein to one or more molecular markers being “detectable” by a molecular marker assay, this means of course that the plant or plant part comprises the one or more markers in its genome, as the marker would otherwise not be detectable.

As used herein, “flanking markers” or “bordering markers” are molecular markers located on the chromosome on either side of an allele or gene of interest, i.e., one marker on the right side of the allele or gene and one marker on the left side of the allele or gene.

As used herein, “closely linked marker” is a marker which is physically close enough to an allele or gene to co-segregate with the allele or gene at a high frequency, i.e., the chance of recombination taking place between the allele or gene and the marker is so small that the marker can be used to reliably select for the presence of the allele or gene in a breeding program (marker-assisted selection).

As used herein, “marker-assisted selection” or “MAS” refers to a process of using the presence of molecular markers, which are genetically and physically linked to a particular locus or to a particular chromosomal region (e.g., introgression fragment), to select plants (e.g., progeny) for the presence of the specific locus or region (e.g., introgression fragment).

As used herein, “marker assay” or “genotyping assay” refers to an assay which can be used to determine the marker genotype, e.g., the SNP genotype. For example, SNP markers can be detected using a KASP-assay or other assays known to the skilled person.

As used herein, the term “phenotype” refers to the detectable characteristics of a cell or organism that can be influenced by gene expression.

As used herein, the term “genotype” refers to the specific allelic makeup of a plant.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, the term “physical distance” referring to a region between loci (e.g., between molecular markers and/or between phenotypic markers) on the same chromosome is the actual physical distance expressed in base pairs (bp), kilobase pairs (kb), or megabase pairs (Mb).

As used herein, the term “genetic distance” between loci (e.g., between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

As used herein, the term “introgressed,” when used in reference to a genetic locus, refers to a genetic locus that has been introduced into a new genetic background, such as through backcrossing. Introgression of a genetic locus can thus be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, refers to markers and/or genomic regions that are located on the same linkage group or chromosome such that they tend to segregate together at meiosis. As used herein, “tolerance locus” means a locus associated with tolerance or resistance to disease. For instance, a tolerance locus according to the present invention may, in one embodiment, control tolerance or susceptibility to ToBRFV or resistance-breaking ToBRFV.

As used herein, “tolerance” or “improved tolerance” in a plant refers to the ability of the plant to perform well, for example by maintaining yield, under disease conditions or upon pest infestations. Tolerance may also refer to the ability of a plant to maintain a plant vigor phenotype under disease conditions or under pest infestations. Tolerance is a relative term, indicating that a “tolerant” plant is more able to maintain performance compared to a different (less tolerant) plant (e.g. a different plant variety) grown in similar disease conditions or under similar pest pressure. One of skill will appreciate that plant tolerance to disease or pest conditions varies widely and can represent a spectrum of more-tolerant or less-tolerant phenotypes. However, by simple observation, one of skill can generally determine the relative tolerance of different plants, plant varieties, or plant families under disease or pest conditions, and furthermore, will also recognize the phenotypic gradations of “tolerance.”

As used herein “resistance” or “improved resistance” in a plant to disease or pest conditions is an indication that the plant is more able to reduce disease or pest burden than a non-resistant or less resistant plant. Resistance is a relative term, indicating that a “resistant” plant is more able to reduce disease burden or pest burden compared to a different (less resistant) plant (e.g., a different plant variety) grown in similar disease conditions or pest pressure. One of skill will appreciate that plant resistance to disease conditions or pest infestation varies widely and can represent a spectrum of more-resistant or less-resistant phenotypes. However, by simple observation, one of skill can generally determine the relative resistance of different plants, plant varieties, or plant families under disease conditions or pest pressure, and furthermore, will also recognize the phenotypic gradations of “resistant.” Resistance can be non-specific or “broad-spectrum” or be race-specific. As used herein, “broad-spectrum resistance to ToBRFV” refers to resistance to both resistance-breaking and non-resistance-breaking ToBRFV isolates.

The terms “percent identity,” “% identity,” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). Sequences that differ by such conservative substitutions are said to have “sequence similarity” or “similarity.” Sequences having a percent identity to a base sequence may exhibit the activity of the base sequence.

As used herein, the term “denoting” when used in reference to a plant genotype refers to any method whereby a plant is indicated to have a certain genotype. This includes any means of identification of a plant having a certain genotype. Indication of a certain genotype may include, but is not limited to, any entry into any type of written or electronic medium or database whereby the plant's genotype is provided. Indications of a certain genotype may also include, but are not limited to, any method where a plant is physically marked or tagged. Illustrative examples of physical marking or tags useful in the invention include, but are not limited to, a barcode, a radio-frequency identification (RFID), a label, or the like.

### VI. Deposit Information

A deposit was made of at least 625 seeds of tomato line FDR-I15-0403V, which comprises the recombinant chromosomal segment on chromosome 11 described herein. The deposit was made with the Provasoli-Guillard National Center for Marine Algae and Microbiota (NCMA), Bigelow Laboratory for Ocean Science, 60 Bigelow Drive, East Boothbay, Me. 04544. The deposit is assigned NCMA Accession No. 202103011, and the date of deposit was Mar. 11, 2021. Access to the deposit will be available during the pendency of the application to persons entitled thereto upon request. The deposit has been accepted under the Budapest Treaty and will be maintained in the NCMA Depository, which is a public depository, for a period of 30 years, or 5 years after the most recent request, or for the enforceable life of the patent, whichever is longer, and will be replaced if nonviable during that period. Applicant does not waive any infringement of their rights granted under this patent or any other form of variety protection, including the Plant Variety Protection Act (7 U.S.C. 2321 et seq.).

A deposit was made of at least 625 seeds of tomato line BVSTBRFVSTACK1, which comprises the Tm1 resistance gene on chromosome 2, the recombinant chromosomal segment on chromosome 11, and the ToBRFV resistance gene on chromosome 8 encoding a ToBRFV resistance protein comprising a polypeptide sequence of SEQ ID NO:19, described herein. The deposit was made with the Provasoli-Guillard National Center for Marine Algae and Microbiota (NCMA), 60 Bigelow Drive, East Boothbay, Maine, 04544 USA. The deposit is assigned NCMA Accession No. 202504007, and the date of deposit was Apr. 17, 2025. Access to the deposit will be available during the pendency of the application to persons entitled thereto upon request. The deposit has been accepted under the Budapest Treaty and will be maintained in the NCMA Depository, which is a public depository, for a period of 30 years, or 5 years after the most recent request, or for the enforceable life of the patent, whichever is longer, and will be replaced if nonviable during that period. Applicant does not waive any infringement of their rights granted under this patent or any other form of variety protection, including the Plant Variety Protection Act (7 U.S.C. 2321 et seq.).

### VII. Further Embodiments

### Plants

E1: A Solanum lycopersicum plant comprising a Tm1 resistance gene on chromosome 2; a recombinant chromosomal segment on chromosome 11, wherein said chromosomal segment comprises a Tomato Brown Rugose Fruit Virus (ToBRFV) resistance allele; and a ToBRFV resistance gene on chromosome 8 encoding a ToBRFV resistance protein comprising a polypeptide sequence of SEQ ID NO:18 or 19; wherein the plant exhibits increased resistance to ToBRFV, including resistance-breaking ToBRFV isolates.
E2: The plant of E1, wherein said ToBRFV resistance allele is further defined as: located within a chromosomal segment flanked by marker locus M1 (SEQ ID NO:1) and marker locus M3 (SEQ ID NO:3) on chromosome 11 in said plant; within a chromosomal segment on chromosome 11 comprising marker locus M2 (SEQ ID NO:2) in said plant; located within a chromosomal segment flanked by marker locus M12 (SEQ ID NO:12) and marker locus M14 (SEQ ID NO:14) on chromosome 8 in said plant; or within a chromosomal segment on chromosome 8 comprising marker locus M13 (SEQ ID NO:13) in said plant.
E3: The plant of E1 or E2, wherein said ToBRFV resistance gene is encoded by a polynucleotide sequence comprising SEQ ID NO:16.
E4: The plant of any one of E1 to E3, wherein a representative sample of seed: comprising said recombinant chromosomal segment has been deposited under NCMA Accession No. 202103011; or comprising the Tm1 resistance gene on chromosome 2, the recombinant chromosomal segment on chromosome 11, and/or the ToBRFV resistance gene on chromosome 8 encoding a ToBRFV resistance protein comprising a polypeptide sequence of SEQ ID NO:19 has been deposited under NCMA Accession No. 202504007.
E5: The plant of any one of E1 to E4, wherein said plant is homozygous for: the Tm1 resistance gene; the ToBRFV resistance allele; or the ToBRFV resistance gene.
E6: The plant of any one of E1 to E5, wherein said plant is homozygous for the Tm1 resistance gene, the ToBRFV resistance allele, and the ToBRFV resistance gene.
E7: The plant of any one of E1 to E6, wherein said resistance-breaking ToBRFV isolate comprises an Asn to Lys substitution at position 82 of a ToBRFV movement protein.
E8: The plant of any one of E1 to E6, wherein said plant comprises resistance to resistance-breaking ToBRFV isolate ToBRFV_G78_RB.
E9: The plant of any one of E1 to E8, wherein said plant exhibits reduced ToBRFV-associated leaf symptoms when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19.
E10: The plant of any one of E1 to E9, wherein said plant exhibits reduced ToBRFV transmissibility when infected with resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19.
E11: The plant of any one of E1 to E10, wherein a plant or plant part exhibits reduced ToBRFV titer when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19.
E12: The plant of any one of E1 to E11, wherein the plant exhibits increased resistance to resistance-breaking ToBRFV isolates during: a crop cycle greater than or equal to about 9, about 8, about 7, about 6, about 5, about 4, or about 3 months; under abiotic stress conditions; or under biotic stress conditions.
E13: The plant of any one of E1 to E12, wherein the resistance to ToBRFV comprises resistance to non-resistance-breaking ToBRFV isolates.
E14: A plant part of a plant of any one of E1 to E13, wherein the plant part is a cell, a seed, a root, a stem, a leaf, a head, a flower, a fruit, or pollen.
E14A: A plant as described in E14, wherein the plant part is a seed or a fruit of said plant.
E14B: A plant as described in E14, wherein the plant part is a seed or a fruit of a plant in accordance with E4, E8, or a combination of E8 with any one of E9 to E13.
E15: A Solanum lycopersicum plant comprising a Tm1 resistance gene on chromosome 2; a recombinant chromosomal segment on chromosome 11, wherein said chromosomal segment comprises a Tomato Brown Rugose Fruit Virus (ToBRFV) resistance allele; and a ToBRFV resistance gene on chromosome 8 encoding a ToBRFV resistance protein comprising a polypeptide sequence of SEQ ID NO:18 or 19; wherein the plant exhibits broad-spectrum resistance to ToBRFV.

### Methods

E16: A method for selecting a tomato plant exhibiting increased resistance to resistance-breaking Tomato Brown Rugose Fruit Virus (ToBRFV) isolates, comprising, crossing a Solanum lycopersicum plant comprising a Tm1 resistance gene on chromosome 2; a recombinant chromosomal segment on chromosome 11, wherein said chromosomal segment comprises a ToBRFV resistance allele; and a ToBRFV resistance gene on chromosome 8 encoding a ToBRFV resistance protein comprising a polypeptide sequence of SEQ ID NO:18 or 19, with itself or with a second tomato plant of a different genotype to produce one or more progeny plants; and selecting a progeny plant comprising the Tm1 resistance gene on chromosome 2, the recombinant chromosomal segment on chromosome 11, and the ToBRFV resistance gene.
E17: The method according to E16, comprising selecting a tomato plant of any one of E1 to E13.
E18: A method for producing a tomato plant exhibiting increased resistance to resistance-breaking Tomato Brown Rugose Fruit Virus (ToBRFV) isolates, comprising introgressing into a plant a Tm1 resistance gene on chromosome 2, a recombinant chromosomal segment on chromosome 11, wherein said chromosomal segment comprises a ToBRFV resistance allele, or a ToBRFV resistance gene on chromosome 8 encoding a ToBRFV resistance protein comprising a polypeptide sequence of SEQ ID NO:18 or 19, wherein the tomato plant exhibits increased resistance to resistance-breaking ToBRFV isolates. In some embodiments, said method comprises introgressing into the plant: the Tm1 resistance gene on chromosome 2; the recombinant chromosomal segment on chromosome 11; the ToBRFV resistance gene on chromosome 8 encoding a ToBRFV resistance protein comprising a polypeptide sequence of SEQ ID NO:18 or 19; or a combination of any thereof.
E19: The method according to E18, wherein said introgressing comprises: crossing a plant comprising the Tm1 resistance gene on chromosome 2, the recombinant chromosomal segment on chromosome 11, and the ToBRFV resistance gene on chromosome 8 with itself or with a second Solanum lycopersicum plant of a different genotype to produce one or more progeny plants; and selecting a progeny plant comprising the Tm1 resistance gene on chromosome 2, the recombinant chromosomal segment on chromosome 11, and the ToBRFV resistance gene on chromosome 8.
E20: The method according to any one of E18 to E19, wherein selecting a progeny plant comprises detecting nucleic acids comprising marker locus M1 (SEQ ID NO:1).
E21: The method according to any one of E18 to E20, wherein selecting a progeny plant comprises detecting nucleic acids comprising marker locus M2 (SEQ ID NO:2).
E22: The method according to any one of E18 to E21, wherein selecting a progeny plant comprises detecting nucleic acids comprising marker locus M3 (SEQ ID NO:3).
E23: The method according to any one of E18 to E22, wherein selecting a progeny plant comprises detecting nucleic acids comprising marker locus M4 (SEQ ID NO:4).
E24: The method according to any one of E18 to E23, wherein selecting a progeny plant comprises detecting nucleic acids comprising marker locus M5 (SEQ ID NO:5).
E25: The method according to any one of E18 to E24, wherein selecting a progeny plant comprises detecting nucleic acids comprising marker locus M6 (SEQ ID NO:6).
E26: The method according to any one of E18 to E25, wherein selecting a progeny plant comprises detecting nucleic acids comprising marker locus M7 (SEQ ID NO:7).
E27: The method according to any one of E18 to E26, wherein selecting a progeny plant comprises detecting nucleic acids comprising marker locus M8 (SEQ ID NO:8).
E28: The method according to any one of E18 to E27, wherein selecting a progeny plant comprises detecting nucleic acids comprising marker locus M9 (SEQ ID NO:9).
E29: The method according to any one of E18 to E28, wherein selecting a progeny plant comprises detecting nucleic acids comprising marker locus M10 (SEQ ID NO:10).
E30: The method according to any one of E18 to E29, wherein selecting a progeny plant comprises detecting nucleic acids comprising marker locus M11 (SEQ ID NO:11).
E31: The method according to any one of E18 to E30, wherein selecting a progeny plant comprises detecting nucleic acids comprising marker locus M12 (SEQ ID NO:12).
E32: The method according to any one of E18 to E31, wherein selecting a progeny plant comprises detecting nucleic acids comprising marker locus M13 (SEQ ID NO:13).
E33: The method according to any one of E18 to E32, wherein selecting a progeny plant comprises detecting nucleic acids comprising marker locus M17 (SEQ ID NO:35).
E34: The method according to any one of E18 to E33, wherein selecting a progeny plant comprises detecting nucleic acids comprising marker locus M14 (SEQ ID NO:14).
E35: A method for controlling or preventing Tomato Brown Rugose Fruit Virus (ToBRFV) symptoms during crop production, wherein the ToBRFV symptoms are caused by resistance-breaking ToBRFV isolates, comprising: obtaining a population of Solanum lycopersicum plants comprising a ToBRFV resistance gene on chromosome 8 encoding a ToBRFV resistance protein comprising a polypeptide sequence of SEQ ID NO:19; growing the Solanum lycopersicum plants; and harvesting fruit of the Solanum lycopersicum plants.
E36: The method according to E35, wherein the population of Solanum lycopersicum plants further comprise: a Tm1 resistance gene on chromosome 2; or a recombinant chromosomal segment on chromosome 11, wherein said chromosomal segment comprises a ToBRFV resistance allele.
E37: The method according to any one of E35 to E36, wherein the population of Solanum lycopersicum plants comprises: the ToBRFV resistance gene on chromosome 8 encoding a ToBRFV resistance protein comprising a polypeptide sequence of SEQ ID NO:19; the Tm1 resistance gene on chromosome 2; and the recombinant chromosomal segment on chromosome 11, wherein said chromosomal segment comprises a Tomato Brown Rugose Fruit Virus (ToBRFV) resistance allele.
E38: A method for improving tomato crop yield in an area comprising resistance-breaking Tomato Brown Rugose Fruit Virus (ToBRFV) isolates, the method comprising: growing a population of Solanum lycopersicum plants comprising a ToBRFV resistance gene on chromosome 8 encoding a ToBRFV resistance protein comprising a polypeptide sequence of SEQ ID NO:19; and harvesting fruit of the Solanum lycopersicum plants, wherein the tomato crop yield of said population of Solanum lycopersicum plants is increased as compared to the yield of a population of Solanum lycopersicum plants lacking the ToBRFV resistance gene.
E39: The method according to E38, wherein the population of Solanum lycopersicum plants further comprise: a Tm1 resistance gene on chromosome 2; or a recombinant chromosomal segment on chromosome 11, wherein said chromosomal segment comprises a ToBRFV resistance allele.
E40: The method according to any one of E38 to E39, wherein the population of Solanum lycopersicum plants comprises: the ToBRFV resistance gene on chromosome 8 encoding a ToBRFV resistance protein comprising a polypeptide sequence of SEQ ID NO:19; the Tm1 resistance gene on chromosome 2; and the recombinant chromosomal segment on chromosome 11, wherein said chromosomal segment comprises a ToBRFV resistance allele.
E41: The method according to any one of E38 to E40, wherein the crop yield of said population of Solanum lycopersicum plants is increased as compared to the yield of a control plant lacking the Tm1 resistance gene, the ToBRFV resistance allele, and the ToBRFV resistance gene on chromosome 8 encoding a ToBRFV resistance protein comprising a polypeptide sequence of SEQ ID NO:19.
E42: The method according to any one of E38 to E41, wherein said resistance-breaking ToBRFV isolate comprises an Asn to Lys substitution at position 82 of a ToBRFV movement protein.
E43: The method according to E38 or E42, wherein said resistance-breaking ToBRFV isolate comprises resistance-breaking ToBRFV isolate ToBRFV_G78_RB.
E44: The method according to any one of E16 to E34, wherein the resistance is determined by inoculating at least one plant to be identified, at least one susceptible control plant, and optionally at least one resistant control plant with ToBRFV inoculums using the following scale with classes 1, 3, 5, 7, and 9, where 1: no symptoms; 3: mild mosaic, mottling hardly visible; 5: clear mosaic symptoms or mild mottling; 7: strong mosaic or clear mottling (leaf packed together); and 9: severe mosaic or strong mottling/misshaped leaf.

Plants can then be scored for disease symptoms, two weeks after inoculation using the above-described scale for Disease Index. The skilled person is well-aware how to determine the Disease Index of a plant. At the time of scoring, minimally 95% of the control plants should show the expected resistant or susceptible reaction.

Usually, an assay is carried out with more than one control plant per genotype, e.g., at least 10 plants, at least 15 plants such as 15 to 60 plants per genotype. When more than one plant of a genotype is used to evaluate the Disease Index, this value will be a mean value of the control plants with the same genotype.
E45: The method according to any one of E16-E34 and E44, wherein the resistance is measured with an assay in accordance with an assay described in Example 1.
E46: A recombinant DNA segment from Solanum lycopersicum comprising a Tomato Brown Rugose Fruit Virus (ToBRFV) resistance allele from Solanum habrochaites that confers increased resistance to ToBRFV, wherein said recombinant DNA segment lacks an allele genetically linked thereto that confers an orange mature fruit color phenotype when present in a plant.
E47: The recombinant DNA segment of E46, wherein said recombinant DNA segment comprises a sequence selected from the group consisting of SEQ ID NOs:12, 13, 14, and 35.
E48: The recombinant DNA segment according to E46 or E47, wherein said recombinant DNA segment comprises the sequence of SEQ ID NO:12.
E49: The recombinant DNA segment according to any one of E46 to E48 wherein said recombinant DNA segment comprises the sequence of SEQ ID NO:13.
E50: The recombinant DNA segment according to any one of E46 to E49, wherein said recombinant DNA segment comprises the sequence of SEQ ID NO:14.
E51: The recombinant DNA segment according to any one of E46 to E50, wherein said recombinant DNA segment comprises the sequence of SEQ ID NO:35.
E52: The recombinant DNA segment according to any one of E46 to E51, wherein said recombinant DNA segment comprises an allele from Solanum lycopersicum at marker locus M12 (SEQ ID NO:12) and marker locus M14 (SEQ ID NO:14) and an allele from Solanum habrochaites at marker locus M13 (SEQ ID NO:13).
E53: The recombinant DNA segment according to any one of E46 to E52, wherein said recombinant DNA segment further comprises an allele from Solanum lycopersicum at marker locus M17 (SEQ ID NO:35).
E54: The recombinant DNA segment according to any one of E46 to E53, further defined as comprised within a plant, plant part, plant cell, or seed.
E55: A method of producing a tomato plant with improved Tomato Brown Rugose Fruit Virus (ToBRFV) resistance, comprising introgressing into said plant at least one ToBRFV resistance allele within a recombinant chromosomal segment flanked in the genome of said plant by marker locus M12 (SEQ ID NO:12) and marker locus M14 (SEQ ID NO:14) on chromosome 8, wherein said introgressed ToBRFV resistance allele confers to said plant increased resistance to ToBRFV compared to a plant not comprising said allele, and wherein said recombinant chromosomal segment lacks an allele genetically linked thereto that confers an orange mature fruit color phenotype when present, and wherein said introgressing comprises marker-assisted selection.
E56: The method according to E55, wherein said introgressing further comprises backcrossing or assaying for said ToBRFV resistance.
E57: The method according to E55 or E56, wherein said introgressing comprises: a) crossing a plant comprising said chromosomal segment with itself or with a tomato plant of a different genotype to produce at least a first progeny plant; and b) selecting a plant comprising said chromosomal segment.
E58: The method according to any one of E55 to E57, wherein marker-assisted selection comprises detecting at least a first marker locus genetically linked to said ToBRFV resistance allele selected from the group consisting of: marker locus M12 (SEQ ID NO:6), marker locus M14 (SEQ ID NO:14), marker locus M17 (SEQ ID NO:35), and marker locus M13 (SEQ ID NO:16).
E59: The method according to any one of E55 to E58, wherein marker-assisted selection comprises detecting marker locus M12 (SEQ ID NO:12).
E60: The method according to any one of E55 to E59, wherein marker-assisted selection comprises detecting marker locus M13 (SEQ ID NO:13).
E61: The method according to any one of E55 to E60, wherein marker-assisted selection comprises detecting marker locus M17 (SEQ ID NO:35).
E62: The method according to any one of E55 to E61, wherein marker-assisted selection comprises detecting marker locus M14 (SEQ ID NO:14).
E63: The method according to any one of E55 to E62, wherein said detecting comprises detecting an allele from Solanum lycopersicum at marker locus M12 (SEQ ID NO:12) and marker locus M14 (SEQ ID NO:14) and an allele from Solanum habrochaites at marker locus M13 (SEQ ID NO:13).
E64: The method according to any one of E55 to E63, further comprising detecting an allele from Solanum lycopersicum at marker locus M17 (SEQ ID NO:35).
E65: A tomato plant obtainable by the method according to any one of E55 to E64.

Embodiments described for plants, methods for producing a plant, methods for selecting a plant in accordance with the present invention described herein can, if applicable, also be used for further defining the methods for identifying a plant in accordance with the present invention.

### EXAMPLES

The following disclosed embodiments are merely representative of the invention which may be embodied in various forms. Thus, specific structural, functional, and procedural details disclosed in the following examples are not to be interpreted as limiting.

### Example 1

### Assays and Protocols for Testing for Resistance-Breaking ToBRFV Resistance

### Bioassay

A screening bioassay was used as described herein to test the infectivity of a newly identified resistance-breaking ToBRFV isolate (Zisi et al. 2024; NCBI GenBank Accession OR760199), to understand infectivity of the new isolate and identify resistant and susceptible cultivars. In brief, the bioassay was conducted as follows: (1) Isolate inoculum prepared from crushing infected leaves in tap water using a mortar and pestle and adding ½ teaspoon carborundum powder per 10 ml to the mix; and (2) Inoculum concentration prepared as 40 gram infected leaf/100 ml tap water and applied via mechanical inoculation on cotyledons of 21 days old plantlets.

The bioassay was conducted in a greenhouse where the light period was set to max 16 hours artificial light, dependent on natural illumination, during which the temperature was minimum 22° C. at day, 20° C. at night. The relative humidity inside the greenhouse was kept 65-85%. Bioassay evaluation was performed 14 (summer)-21 (winter) days post inoculation, based on minimum 90% symptom expression on controls. ToBRFV symptom scoring was performed at either 14 or 21 days post inoculation (DPI) according to a scoring scale with classes 1, 3, 5, 7, and 9 where 1: no symptoms; 3: mild mosaic, mottling hardly visible; 5: clear mosaic symptoms or mild mottling; 7: strong mosaic or clear mottling (leaf packed together); and 9: severe mosaic or strong mottling/misshaped leaf.

Quantitative Testing (rt-QPCR)

25 mg of leaf tissue was homogenized in 700 mL GH⁺ buffer (6M Guanidine hydrochloride; 0.2M Sodium acetate; 25 mM EDTA; 2.5% w/v PVP-10). 5 mm metallic beads were added to the sample tubes and using a paint shaker in standard cycle of 3 minutes, samples were homogenized. 200 uL clear sample was transferred to the MPLC96 Processing Cartridge, and using the Roche MagNA Pure 96 DNA and Viral NA SV Kit, RNA was purified In the Roche MagNA Pure 96 equipment.

Samples were tested using a TaqMAN based quantitative RT-qPCR protocol. ToBRFV primer and probe sequences, as well as the RT-qPCR reaction volumes, were adapted from the protocol of Menzel & Winter., 2019. 8 uL master mix was aliquoted into each well in an Applied Biosystems™ MicroAmp™ Optical 96-Well Reaction Plate. 2 ul sample was added to each well, and the plates were sealed using Applied Biosystems™ MicroAmp™ Optical Adhesive Film. Plates were loaded into the QuantStudio™ 6 Pro Real-Time PCR System device. Protocol for qPCR as follows: 1) Reverse transcription (RT) reaction at 48° C. for 5 minutes; followed by 2) RT inactivation reaction at 95° C. for 20 seconds; followed by 3) 2-step fast-mode PCR reaction of 95° C. for 3 seconds, 60° C. for 20 seconds for 40 cycles. Fluorescence data was collected at the end of each PCR cycle.

TABLE 3
RT-qPCR primer and probe sequences

| Target | Primer name | Sequence |
|---|---|---|
| ToBRFV | Menzel Fw | 5′-CAATCAGAGCACATTTGAA |
| (From Menzel & | | AGTGCA-3′ |
| Winter., 2019) | Menzel Rv | 5′-CAGACACAATCTGTTATTT |
| | | AAGCATC-3′ |
| | ModMenzel Pr | FAM 5′-ACAATGGTCCTCTGC |
| | | ACCTG-3′ IBFQ |
| Tip41 | ICG-Fw | 5′-AACAGGTGGTGCTCGACTA |
| | | TGACT-3′ |
| | ICG-Rv | 5′-TGCTTTCGACAGTTTCACT |
| | | TCCA-3′ |
| | ICG-Pr | CY5 5′-ACCTTCACAACACCT |
| | | TACT-3′ IBFQ |

Using Design and Analysis software, provided by the manufacturer, Relative quantity (RQ) was calculated through the ddCT method. Susceptible control sample from each trial was used as the Susceptible reference for that sample set. Samples were normalized using an internal control gene (ICG), i.e. housekeeping gene. The final relative quantity data RQ was exported and used for further analysis.

### Example 2

### Allelic Variation in ToBRFV Resistance Gene on Chromosome 8 is Associated with Differential Phenotypic Expression

Solanum habrochaites accession LA2812 has been suggested as a source of resistance to ToBRFV (Jewehan, A., et al., “Evaluation of responses to tomato brown rugose fruit virus (ToBFRV) and resistance line in Solanum habrochaites and Solanum peruvianum germplasm,” Viral and Viroid Diseases, 88:187-196 (2022)). Furthermore, Ykema (WO2020/148021) suggests an allele on chromosome 8 as a causal resistance gene. This example describes previously unknown resistance to resistance-breaking ToBRFV isolates associated with allelic variation in the resistance gene described by Ykema. In particular, various alleles on chromosome 8 having differences of one to several nucleotides are presented. Surprisingly, these relatively small differences at DNA and protein level yield significant phenotypic variation in resistance behavior. Provided herein is a method to distinguish between the various alleles and the unexpected identification of an allele conferring improved resistance to resistance-breaking ToBRFV.

### Identification of LA2812 as a Resistance Source to ToBRFV

580 accessions were screened for resistance to ToBRFV. LA2812 was identified as an accession with individual plants with high resistance to ToBRFV. Some plants were also susceptible. (Tomato Genetics Resource Center Accession LA2812 https://tgrc-mvc.plantsciences.ucdavis.edu/Accession/detail/LA2812) This result indicates that the accession segregates for ToBRFV resistance.

Creating Resistance Donors from Resistance Source LA2812

Accession LA2812 segregates for resistance to ToBRFV. Through infecting and phenotyping cuttings from individual plants, these plants were identified as resistant (LA2812-1 through LA2812-6) or susceptible (LA2812-7 and LA2812-8) to ToBRFV.

### Mapping LA2812 Resistance to QTL8

LA2812-1 was crossed to a breeding line susceptible to ToBRFV (Inbred_A). Through cuttings, the individual F1 plants were confirmed to be resistant to ToBRFV (resistance was inherited) and they were self-pollinated to generate F2 populations.

143 F2 plants were phenotyped for ToBRFV resistance and genotyped across the whole genome with 117 markers that are polymorphic between LA2812 and Inbred_A. QTL mapping was performed with the resultant data using standard interval mapping. A significant peak was identified on chromosome 8. This peak co-located with that described by Ykema et al.

### Gene Sequencing in QTL8 Shows Three Distinct Alleles

Genome assemblies, phased per haplotype, of three resistant plants (LA2812-2, LA2812-3 and LA2812-4) were performed. Phased haplotypes were used because these plants likely contain significant amounts of heterozygosity and this method can distinguish the exact sequences at the regions of interest. In these three plants three unique haplotypes were identified as described in Table 4. Three alleles were distinguished using this method, one of them contains the sequence described by Ykema et al (referred to as QTL8A). A second allele is different from this first allele at a single nucleotide as described by Jewehan et al (referred to as QTL8B). A third allele is novel and more distinct from the previous two (referred to as QTL8C), having 96% sequence identity to SEQ ID NO:15. QTL8C likely does not result in a functional polypeptide as it comprises several stop codons within the coding sequence.

TABLE 4
Haplotypes Present in Three ToBRFV Resistant Plants.

| Plant | ToBRFV Phenotype | Phased genome | Haplotype at QTL8 |
|---|---|---|---|
| LA2812-2 | Resistant | LA2812-2-h1 | QTL8A |
| | | LA2812-2-h2 | QTL8B |
| LA2812-3 | Resistant | LA2812-3-h1 | QTL8B |
| | | LA2812-3-h2 | QTL8B |
| LA2812-4 | Resistant | LA2812-4-h1 | QTL8B |
| | | LA2812-4-h2 | QTL8C |

A targeted sequencing approach was developed to further understand which of these haplotypes are associated with resistance to ToBRFV. Specific PCR products of ~3.5 Kb were amplified from 100 LA2812 plants and sequenced using Pac-Bio long read sequencing. This allowed accurate identification of specific alleles present in a single plant. These same 100 LA2812 plants were also assessed for ToBRFV resistance. The association of the haplotypes to resistance is summarized in Table 5. It is noted that two alleles (QTL8A and QTL8B) provide resistance to ToBRFV and one does not (QTL8C).

TABLE 5
ToBRFV Phenotype Observed in Plants Homozygous
for QTL8A, QTL8B, and QTL8C.

| Allele | ToBRFV Phenotype | SEQ ID NO |
|---|---|---|
| QTL8A | Resistant | 15 |
| QTL8B | Resistant | 16 |
| QTL8C | Susceptible | 17 |

### Example 3

### QTL8B Confers Resistance to Resistance-Breaking ToBRFV

Previous studies have demonstrated that the QTL8 from Ykema et al. (QTL8A) does not provide resistance to resistance-breaking ToBRFV (Zisi et al., “Single amino acid change in tomato brown rugose fruit virus breaks virus-specific resistance in new resistant tomato cultivar,” Frontiers Plant Science, vol. 15 May 2024). A second allele of the gene underlying QTL8 resistance has been described by Jewehan et al. This second allele (QTL8B) was not experimentally demonstrated to provide resistance to ToBRFV although the accession in which it was found is resistant to standard ToBRFV (Jewehan et al). Given the high degree of sequence similarity between QTL8A and QTL8B (99%), QTL8B would likewise not be expected to provide resistance to resistance-breaking ToBRFV.

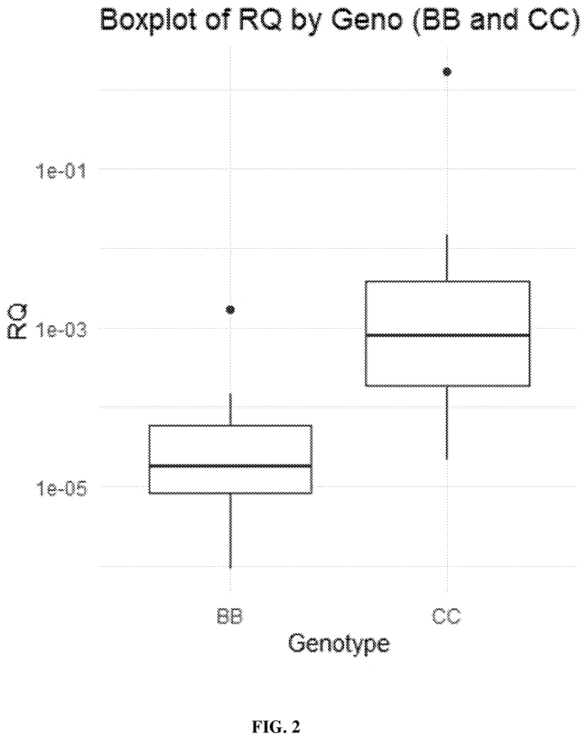

To further investigate resistance to resistance-breaking ToBRFV conferred by QTL8B and QTL8C, a population of 100 LA2812 plants, known to segregate for QTL8A, B and C were infected with resistance-breaking ToBRFV. Targeted sequencing was used to then identify the underlying alleles present in each of the 100 plants. It was found that while QTL8A and QTL8C do not provide resistance to resistance-breaking ToBRFV, QTL8B surprisingly does. That is, a single amino acid change (K223E) in the protein encoded by the ToBRFV resistance gene on chromosome 8 (QTL8B) yields resistance to resistance-breaking ToBRFV as compared to QTL8A. Additionally, it was demonstrated that in LA2812, individuals fixed for QTL8B accumulate significantly less virus than individuals fixed for QTL8C (FIG. 2).

To further confirm this observation, LA2812 plants known to contain each of the three alleles were crossed to a cherry tomato line, Inbred_E, which is phenotypically susceptible to ToBRFV. Individual F₁ plants identified to contain QTL8A, QTL8B or QTL8C were independently back-crossed to this same recurrent parent for 3 generations. At each backcross generation, plants were selected for QTL8 using marker loci M12 (SEQ ID NO:12), M13 (SEQ ID NO:13), and M14 (SEQ ID NO:14) which confirm the presence of the introgression, regardless of the QTL8 allele (A, B, or C). The presence of the respective QTL8 alleles (A, B, or C) was tracked by following the parentage of the individual plants. Alternatively, the presence of the respective QTL8 alleles may have also been tracked by direct sequencing. For each backcross family, individual plants identified as heterozygous for their respective allele (QTL8A, B, or C) were self-pollinated to produce BC₃F₃ families.

A BC3F2 family for each of the three alleles was sown and 15 plants that were homozygous fixed were selected for each of the alleles at QTL8 (using markers locus M12, M13, and M14 as before). A bioassay for resistance-breaking ToBRFV was performed on these plants in a randomized block design, with 5 plants per block of each allele at QTL8. Susceptible checks (Inbred_A and plants grown from NCIMB43279, comprising QTL8A) were added to the experimental design. As can be seen in Table 6 below, QTL8B surprisingly confers improved resistance to resistance-breaking ToBRFV as compared to QTL8A and QTL8C. That is, the presence of a single amino acid substitution is unexpectedly associated with resistance to resistance-breaking ToBRFV isolates.

TABLE 6
Resistance-breaking ToBRFV Phenotype Observed in
Plants Homozygous for QTL8A, QTL8B, and QTL8C.

| | ToBRFV | Resistance-breaking | |
|---|---|---|---|
| Allele | Phenotype | ToBRFV Phenotype | SEQ ID NO |
| QTL8A | Resistant | Susceptible | 15 |
| QTL8B | Resistant | Resistant | 16 |
| QTL8C | Susceptible | Susceptible | 17 |

### Example 4

### High-Level Resistance to rbToBRFV Through Synergetic Effects of Stacked QTLs

Zisi et al. recently reported and characterized a resistance-breaking ToBRFV strain. Specifically, they reported that commercial varieties claimed to have ToBRFV resistance genes on chromosome 8 are susceptible to this resistance-breaking ToBRFV isolate. In view of this, a range of available sources of resistance were challenged with this resistance-breaking ToBRFV isolate and their behavior was investigated. As shown in FIG. 1, none of the sources show symptomless behavior (phenotype=1) in combination with strongly reduced virus multiplication (Log(RQ)<−5). Varieties with QTL11 (i.e. a recombinant chromosomal segment on chromosome 11, wherein said chromosomal segment comprises a Tomato Brown Rugose Fruit Virus (ToBRFV) resistance allele) and Tm1 (in homozygous or heterozygous condition) show a trend towards fewer symptoms compared to the susceptible controls at 14 and 21 DPI, the difference being more pronounced at 14 DPI. Varieties with QTL11 and Tm1 in homozygous condition show significantly reduced virus multiplication compared to susceptible controls and NCIMB43279, while there is a trend to reduce virus multiplication compared to varieties with Tm-1 in heterozygous condition (FIG. 1). As such, none of the currently existing sources of resistance can be used to completely overcome the increasing problems caused by resistance-breaking ToBRFV.

As described in Example 3, QTL8B surprisingly confers resistance to resistance-breaking ToBRFV where QTL8A and QTL8C do not confer such resistance. To determine if a plant comprising a Tm1 resistance gene on chromosome 2, a recombinant chromosomal segment on chromosome 11, wherein said chromosomal segment comprises a Tomato Brown Rugose Fruit Virus (ToBRFV) resistance allele, and a QTL8B resistance gene on chromosome 8 is capable of reducing virus multiplication and stopping transmission of resistance-breaking ToBRFV to susceptible tomato plants, a fresh market tomato line (Inbred_B) susceptible to ToBRFV and resistance-breaking ToBRFV was used as a recurrent parent in two independent backcrossing experiments for the following QTLs: QTL8B (from LA2812-3), and Tm1 and QTL11 (from an elite parent Inbred_D). Each trait was selected at each backcross generation using the markers described herein. The Tm1 and QTL11 BC₃ plants were self-pollinated and a plant comprising both QTLs was brought to a homozygous state one generation further, referred to as FIR-Tm1-QTL11. QTL8B BC₂ plants were crossed to plants of FIR-Tm1-QTL11. Progeny containing all three resistance loci were self-pollinated. The resulting population, FIR-Tm1-QTL11-QTL8B, segregates for all three QTLs. Following this method, a population where three resistance QTL are segregating within the same genetic background was obtained, enabling conclusions on the effect of resistance loci under study without confounding influence of background genetics.

TABLE 7
Genotypic groups resultant from a backcross experiment that
will be tested for resistance-breaking ToBRFV resistance.

| FIR-Tm1-QTL11-QTL8B geno-group | Tm1 | QTL11 | QTL8B |
|---|---|---|---|
| 1 | +/+ | +/+ | −/− |
| 2 | +/+ | +/+ | +/− |
| 3 | +/+ | +/+ | +/+ |
| 4 | +/− | +/+ | +/− |
| 5 | +/− | +/+ | +/+ |
| 6 | +/− | +/+ | −/− |
| 7 | −/− | −/− | +/+ |

The FIR-Tm1-QTL11-QTL8B (encoding SEQ ID NO:19) population will be sown and 15 plants of the genotypes listed in Table 7 will be selected. These plants will be infected with resistance-breaking ToBRFV and phenotyped, according to the bioassay protocol described in Example 1 in a randomized block design. Susceptible checks (Inbred_A, Inbred_B and/or plants grown from NCIMB43279, comprising QTL8A) will also be included.

Plants homozygous for the recombinant chromosomal segment on chromosome 11 and heterozygous for the Tm1 gene exhibit reduced mosaic symptoms in plant seedlings, along with reduced virus accumulation, when exposed to resistance-breaking ToBRFV. Symptoms appear to be slightly improved when Tm1 and the recombinant chromosomal segment on chromosome 11 are both present homozygously. The presence of Tm1 in combination with the recombinant chromosomal segment on chromosome 11 appears to further improve resistance to mosaic symptoms in seedlings when compared to the homozygous recombinant chromosomal segment on chromosome 11 alone (FIG. 1). Based on the surprising results regarding QTL8B described in Example 3, plants comprising the three resistance loci (Tm1-QTL11-QTL8B) will be evaluated for reduced leaf symptoms in seedlings as compared to plants comprising Tm1-QTL11 or QTL8B; significantly reduced virus accumulation in seedlings as compared to plants comprising Tm1-QTL11 or QTL8B; and significantly reduced seedling virus transmissibility as compared to plants comprising Tm1-QTL11 or QTL8B.

A virus transmissibility study was conducted to evaluate the ability of the resistance loci described above to reduce transmission of rbToBRFV to susceptible plants. Genotype groups consisted of plants containing no resistance loci, only QTL8A (plants from NCIMB number 43279), only QTL8B, the combination of Tm1 and QTL11, and the combination of Tm1, QTL11, and QTL8B. Three plants per genotype group were inoculated with rbToBRFV. After 28 days, tissue from each plant was used to inoculate 12 susceptible Inbred_A plants, resulting in a total of 36 inoculated susceptible plants per genotype. At 21 days post inoculation, tissue from each of the 36 inoculated plants were evaluated for viral load. The results are shown in Table 8 below as percent of susceptible plants categorized as having virus transmitted is presented.

TABLE 8
Summary of results from the transmissibility study
on different QTL combinations (genotype groups)
derived from the FIR-Tm1-QTL11-QTL8B population.

| | # Inbred_A Plants w/ | Percent Inbred_A Plants |
|---|---|---|
| Genotype Group | Virus Transmission | Virus Transmitted |
| Tm1-QTL11-QTL8B | 8 | 22% |
| QTL8B | 16 | 44% |
| Tm1-QTL11 | 24 | 66% |
| QTL8A | 29 | 80% |
| Null | 36 | 100% |

Table 8 shows that plants containing none of the three QTLs (Null) lead to transmission to 100% (36 plants) of susceptible plants. Plants containing only QTL8A or only QTL8B lead to transmission to 80% or 44% of susceptible plants, respectively. Plants containing the combination of Tm1 and QTL11 reduce transmission to 66% of susceptible plants. The greatest reduction in transmission was observed from plants containing the combination of Tm1, QTL11, and QTL8B, where transmission was confirmed in only 22% of susceptible plants. This combination is likely to significantly reduce transmission in a grower environment when rbToBRFV is present or is introduced into that environment.

The mean RQ for Tm1-QTL11-QTL8B trended lower than either Tm1-QTL11 or QTL8B (data not shown). The RQ value for genotype groups Tm1-QTL11, QTL8B, and Tm1-QTL11-QTL8B were not significantly different from each. Samples with an RQ value smaller than 1×10⁻⁵ were considered to have 100,000× less virus than a susceptible plant. Plants above this threshold were categorized as having virus transmitted.

### Example 5

### Enhanced Fruit Quality with a Minimal Introgression of QTL8B

To identify a suitable source for QTL8B for use in breeding programs, various reduced introgressions were generated based on the original mapping interval. Plants were screened for recombinants between markers M15 and M13, where the resistance was likely retained. These recombinants were brought to the BC₃ generation and plants were screened for recombinants between markers M13 and M18, where the resistance was likely retained. Nine favorable recombinants were further backcrossed. At the BC₄ generation, plants from each of the nine recombinant families were genotyped with markers M12-M18. Four distinct haplotypes that likely retained rbToBRFV resistance were identified. The individual plants from these families were selfed to obtain BC₄F₂ seed.

A trial was set up to evaluate differences in agronomic performance and produce quality between the different introgression sizes represented by the four haplotypes. For each BC₄F₂ family, 21 plants that were homozygous for the introgression and 21 plants that were homozygous for the absence of the introgression were selected. A randomized blocked design with three replications of seven plants per genotype was planted, with split-plots to ensure the alternate genotypes were adjacent to each other in each replication. The trial was evaluated for several agronomic and quality features, including mature fruit color. The phenotypic data from the trial was analyzed to identify characteristics that were significantly associated with the different introgression sizes. Within each family the effect of the introgression was compared to the sibling plants that did not contain the introgression to control for background effects from backcrossing.

Fruit color at maturity was defined according to the guidance from the Calibration manual for DUS Test for tomato based on CPVO protocol TP/44/4 and UPOV guideline TG/44/11. The results of the mature fruit color evaluation are shown in Table 9 below.

TABLE 9
Overview of the recombinant haplotypes generated at QTL8B and mature fruit color phenotype.

| Marker | | M15 | M16 | M12 | M13 | M17 |
|---|---|---|---|---|---|---|
| Genetic position | | 57.68 | 60.67 | 61.01 | 61.40 | 62.01 |
| (cM)* | | | | | | |
| Position (bp) | | 59,306,115 | 59,673,267 | 59,715,652 | 59,762,442 | 59,836,842 |
| SL2.50** | | | | | | |
| QTL8B | 1 | TT | GG | GG | TT | AA |
| Haplotypes | 2 | TT | GG | GG | TT | AA |
| | 3 | TT | AA | CC | TT | AA |
| | 4 | TT | AA | CC | TT | CC |
| | LA2812 | AA | GG | GG | TT | AA |
| | Inbred B | TT | AA | CC | CC | CC |

| Marker | | M14 | M18 | # families | Fruit Color |
|---|---|---|---|---|---|
| Genetic position | | 63.11 | 63.68 | | |
| (cM)* | | | | | |
| Position (bp) | | 59,968,063 | 60,038,311 | | |
| SL2.50** | | | | | |
| QTL8B | 1 | TT | CC | 2 | Orange |
| Haplotypes | 2 | CC | CC | 3 | Orange |
| | 3 | CC | CC | 1 | Red |
| | 4 | CC | CC | 3 | Red |
| | LA2812 | TT | TT | 1 | Orange |
| | Inbred B | CC | CC | 1 | Red |

*Inferred from the SL2.50 physical assembly from genetic map data
**with reference to tomato genome physical assembly version SL2.50

In Table 9 above, haplotypes 1 and 2 were associated with an undesirable orange mature fruit color across the five independent recombinant families. In contrast, plants comprising either of the two smaller introgressions, represented by haplotypes 3 and 4, did not have orange mature fruit. Instead, they possessed the same red mature fruit color as plants of Inbred_B and plants of their sibling lines that do not contain the introgression. The results demonstrate a clear effect of introgression size on the color of mature fruits.

Through disease assays, it was observed that resistance to rbToBRFV was retained in plants containing any of haplotypes 1 through 4 (data not shown).

The orange mature fruit color was found to be in strong linkage with QTL8B however this drag can be uncoupled with the use of specific makers. To remove the undesirable fruit color trait while maintaining resistance, recombination is required between markers M12 and M13, such that the donor allele (derived from LA2812) is present at marker M13 and the non-donor allele (derived from Solanum lycopersicum) is present at marker M12. To further reduce the introgression, a further recombination event should be selected for, such that a non-donor allele is present at marker M14. A donor allele can be present at marker M17 however a non-donor allele at marker M17 is preferred for a minimal introgression with donor DNA.

### Example 6

### Obtaining Durable Resistance to ToBRFV Through Stacked Resistance QTLs

ToBRFV is virus that has a high mutation rate and can thus overcome previously effective genetic resistance. It is a continuing need to identify means for conferring durable genetic resistance. An experiment was conducted to determine if any combination(s) of the Tm1 locus, QTL11, and QTL8B conferred durable resistance to ToBRFV.

Plants derived from the FIR-Tm1-QTL11-QTL8B population described above were used for two different screening bioassays. The tested plants either had no resistance QTLs (Null), QTL8B alone (QTL8B), the Tm1 locus and QTL11 (Tm1-QTL11), or the Tm1 locus, QTL11, and QTL8B (Tm1-QTL11-QTL8B) In one bioassay, plants of each genotype were exposed to the rbToBRFV isolate OR760199 and in the other bioassay, plants of each genotype were exposed to the non-rbToBRFV isolate VIR351. Virus accumulation was measured 24 days post inoculation (DPI). The results are shown in Table 10 below.

TABLE 10
Virus accumulation measured 24 DPI for different resistance QTL
combinations derived from the FIR-Tm1-QTL11-QTL8B population.

| | rbToBRFV Isolate OR760199 | | ToBRFV Isolate VIR351 | |
|---|---|---|---|---|
| | | Signif- | | Signif- |
| Genotype | Median RQ | icance | Median RQ | icance |
| Group | 24 DPI | group* | 24 DPI | group* |
| Null | 1.18 | A | 0.245 | A |
| QTL8B | 2.76 × 10⁻² | B | 9.18 × 10⁻⁷ | B |
| Tm1-QTL11 | 1.75 × 10⁻⁴ | C | 0.299 | A |
| Tm1-QTL11- | 9.54 × 10⁻⁵ | D | 4.63 × 10⁻⁷ | B |
| QTL8B | | | | |

*Significance group letters represent pairwise Wilcoxon rank-sum tests (BH-adjusted, α = 0.05).

Table 10 shows that in the rbToBRFV bioassay, the resistance level conferred by QTL8B was significantly greater than the Null, while the Tm1-QTL11 double stack demonstrated the second largest effect. The Tm1-QTL11-QTL8B triple stack provided the largest effect on improved resistance, which was approximately an order of magnitude larger than the double stack effect. In contrast, in the non-rbToBRFV bioassay, the Tm1-QTL11 double stack provided no significant reduction in virus accumulation compared to the Null. QTL8B and the triple stack provided an equally significant reduction in virus accumulation compared to the Null.

Furthermore, the VIR351 isolate was found to be more virulent compared to another non-rbToBRFV isolate where all resistance QTL combinations shown in Table 10 performed equally when infected with this other non-rbToBRFV isolate (data not shown).

Together, these results demonstrate that different genetics are needed for resistance to different ToBRFV isolates and that combining resistance QTLs provides improved consistent resistance across ToBRFV isolates. The combination of different modes of action for resistance conferred by QTL8B and Tm1-QTL11 was shown to provide durable resistance to ToBRFV. This durable resistance provides greater protection for plants in any growing environment by conferring resistance via different modes of action to a variety of resistance-breaking or non-resistance-breaking ToBRFV isolates that may be present in a growing environment.

1. A Solanum lycopersicum plant comprising:
(a) a Tm1 resistance gene on chromosome 2;
(b) a recombinant chromosomal segment on chromosome 11, wherein said chromosomal segment comprises a Tomato Brown Rugose Fruit Virus (ToBRFV) resistance allele; and
(c) a ToBRFV resistance gene on chromosome 8 encoding a ToBRFV resistance protein comprising a polypeptide sequence of SEQ ID NO:18 or 19;
wherein the plant exhibits increased resistance to ToBRFV, including to resistance-breaking ToBRFV isolates.

2. The plant of claim 1, wherein:
(a) said ToBRFV resistance allele is further defined as:
(i) located within a chromosomal segment flanked by marker locus M1 (SEQ ID NO:1) and marker locus M3 (SEQ ID NO:3) on chromosome 11 in said plant; or
((ii) within a chromosomal segment on chromosome 11 comprising marker locus M2 (SEQ ID NO:2) or marker locus M11 (SEQ ID NO:11) in said plant; or
(b) said ToBRFV resistance gene on chromosome 8 is further defined as:
(i) located within a chromosomal segment flanked by marker locus M12 (SEQ ID NO:12) and marker locus M14 (SEQ ID NO:14) on chromosome 8 in said plant; or
(ii) within a chromosomal segment on chromosome 8 comprising marker locus M13 (SEQ ID NO:13) in said plant.

3. The plant of claim 1, wherein:
(a) said ToBRFV resistance gene is encoded by a polynucleotide sequence comprising SEQ ID NO:16;
(b) said resistance-breaking ToBRFV isolate comprises an Asn to Lys substitution at position 82 of a ToBRFV movement protein;
(c) said plant comprises resistance to resistance-breaking ToBRFV isolate Vir353 (NCBI GenBank Accession OR760199);
(d) said plant exhibits reduced ToBRFV-associated leaf symptoms when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19;
(e) said plant exhibits reduced ToBRFV transmissibility when infected with resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19
(f) a fruit of said plant exhibits reduced ToBRFV titer when exposed to resistance-breaking ToBRFV isolates as compared to a control plant lacking the Tm1 resistance gene, the ToBRFV resistance allele, and a ToBRFV resistance gene on chromosome 8 encoding a ToBRFV resistance protein comprising a polypeptide sequence of SEQ ID NO:19; or
(g) a representative sample of seed comprising:
(i) said recombinant chromosomal segment has been deposited under NCMA Accession No. 202103011; or
(ii) the Tm1 resistance gene on chromosome 2, the recombinant chromosomal segment on chromosome 11, or the ToBRFV resistance gene on chromosome 8 encoding a ToBRFV resistance protein comprising a polypeptide sequence of SEQ ID NO:19 has been deposited under NCMA Accession No. 202504007.

4. (canceled)

5. The plant of claim 1, wherein said plant is homozygous for:
(a) the Tm1 resistance gene;
(b) the ToBRFV resistance allele; or
(c) the ToBRFV resistance gene.

6. The plant of claim 5, wherein said plant is homozygous for the Tm1 resistance gene, the ToBRFV resistance allele, and the ToBRFV resistance gene.

7.-11. (canceled)

12. The plant of claim 1, wherein the plant exhibits increased resistance to resistance-breaking Tomato Brown Rugose Fruit Virus (ToBRFV) isolates during:
(a) a crop cycle greater than or equal to about 8 months;
(b) under abiotic stress conditions; or
(c) under biotic stress conditions.

13. A cell, seed, or plant part of the plant of claim 1, wherein the cell, seed, or plant part comprises the Tm1 resistance gene, the ToBRFV resistance allele, and the ToBRFV resistance gene.

14. (canceled)

15. A method of selecting a tomato plant exhibiting increased resistance to resistance-breaking Tomato Brown Rugose Fruit Virus (ToBRFV) isolates, comprising:
a) crossing the tomato plant of claim 1 with itself or with a second tomato plant of a different genotype to produce one or more progeny plants; and
b) selecting a progeny plant comprising the Tm1 resistance gene on chromosome 2, the recombinant chromosomal segment on chromosome 11, and the ToBRFV resistance gene.

16. A method of producing a tomato plant exhibiting increased resistance to resistance-breaking Tomato Brown Rugose Fruit Virus (ToBRFV) isolates, comprising introgressing into a plant a Tm1 resistance gene on chromosome 2, a recombinant chromosomal segment on chromosome 11, wherein said chromosomal segment comprises a ToBRFV resistance allele, or a ToBRFV resistance gene on chromosome 8 encoding a ToBRFV resistance protein comprising a polypeptide sequence of SEQ ID NO:18 or 19, wherein the tomato plant exhibits increased resistance to resistance-breaking ToBRFV isolates.

17. The method of claim 16, wherein said method comprises introgressing into the plant:
the Tm1 resistance gene on chromosome 2;
the recombinant chromosomal segment on chromosome 11;
the ToBRFV resistance gene on chromosome 8 encoding a ToBRFV resistance protein comprising a polypeptide sequence of SEQ ID NO:18 or 19; or
a combination of any thereof.

18. The method of claim 16, wherein said introgressing comprises:
a) crossing a plant comprising the Tm1 resistance gene on chromosome 2, the recombinant chromosomal segment on chromosome 11, and the ToBRFV resistance gene on chromosome 8 with itself or with a second Solanum lycopersicum plant of a different genotype to produce one or more progeny plants; and
b) selecting a progeny plant comprising the Tm1 resistance gene on chromosome 2, the recombinant chromosomal segment on chromosome 11, and the ToBRFV resistance gene on chromosome 8.

19. The method of claim 16, wherein:
(a) a sample of seed comprising said recombinant chromosomal segment has been deposited under NCMA Accession No. 202103011;
(b) selecting a progeny plant comprises detecting nucleic acids comprising marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), M12 (SEQ ID NO:12), M13 (SEQ ID NO:13), M17 (SEQ ID NO:35), or M14 (SEQ ID NO:14);
(c) said crossing comprises backcrossing, marker-assisted selection, or assaying for said ToBRFV resistance; or
(d) the progeny plant is an F₂-F₆ progeny plant.

20.-23. (canceled)

24. A method for controlling or preventing ToBRFV symptoms during crop production, wherein the ToBRFV symptoms are caused by resistance-breaking Tomato Brown Rugose Fruit Virus (ToBRFV) isolates, comprising:
a) obtaining a population of Solanum lycopersicum plants comprising a ToBRFV resistance gene on chromosome 8 encoding a ToBRFV resistance protein comprising a polypeptide sequence of SEQ ID NO:19;
b) growing the Solanum lycopersicum plants; and
c) harvesting fruit of the Solanum lycopersicum plants.

25. The method of claim 24, wherein the population of Solanum lycopersicum plants further comprise:
a Tm1 resistance gene on chromosome 2; or
a recombinant chromosomal segment on chromosome 11, wherein said chromosomal segment comprises a ToBRFV resistance allele.

26. The method of claim 25, wherein the population of Solanum lycopersicum plants comprises:
the ToBRFV resistance gene on chromosome 8 encoding a ToBRFV resistance protein comprising a polypeptide sequence of SEQ ID NO:19;
the Tm1 resistance gene on chromosome 2; and
the recombinant chromosomal segment on chromosome 11, wherein said chromosomal segment comprises a ToBRFV resistance allele.

27. A method for improving tomato crop yield in an area comprising resistance-breaking Tomato Brown Rugose Fruit Virus (ToBRFV) isolates, the method comprising:
a) growing a population of Solanum lycopersicum plants comprising a ToBRFV resistance gene on chromosome 8 encoding a ToBRFV resistance protein comprising a polypeptide sequence of SEQ ID NO:19; and
b) harvesting fruit of the Solanum lycopersicum plants, wherein the tomato crop yield of said population of Solanum lycopersicum plants is increased as compared to the yield of a population of Solanum lycopersicum plants lacking the ToBRFV resistance gene.

28. The method of claim 27, wherein the population of Solanum lycopersicum plants further comprise:
a Tm1 resistance gene on chromosome 2; or
a recombinant chromosomal segment on chromosome 11, wherein said chromosomal segment comprises a ToBRFV resistance allele.

29. The method of claim 27, wherein the population of Solanum lycopersicum plants comprises:
the ToBRFV resistance gene on chromosome 8 encoding a ToBRFV resistance protein comprising a polypeptide sequence of SEQ ID NO:19;
the Tm1 resistance gene on chromosome 2; and
the recombinant chromosomal segment on chromosome 11, wherein said chromosomal segment comprises a ToBRFV resistance allele.

30. The method of claim 27, wherein:
(a) the crop yield of said population of Solanum lycopersicum plants is increased as compared to the yield of a population of control plants lacking the Tm1 resistance gene, the ToBRFV resistance allele, and a ToBRFV resistance gene on chromosome 8 encoding a ToBRFV resistance protein comprising a polypeptide sequence of SEQ ID NO:19;
(b) said resistance-breaking ToBRFV isolate comprises an Asn to Lys substitution at position 82 of a ToBRFV movement protein; or
(c) said resistance-breaking ToBRFV isolate comprises resistance-breaking ToBRFV isolate Vir353 (NCBI GenBank Accession OR760199).

31.-32. (canceled)

33. A recombinant DNA segment from Solanum lycopersicum comprising a Tomato Brown Rugose Fruit Virus (ToBRFV) resistance allele from Solanum habrochaites that confers increased resistance to ToBRFV, wherein said recombinant DNA segment lacks an allele genetically linked thereto that confers an orange mature fruit color phenotype when present in a plant.

34. The recombinant DNA segment of claim 33:
(a) wherein said recombinant DNA segment comprises a sequence selected from the group consisting of SEQ ID NOs:12, 13, 14, and 35;
(b) wherein said recombinant DNA segment comprises an allele from Solanum lycopersicum at marker locus M12 (SEQ ID NO:12) and marker locus M14 (SEQ ID NO:14) and an allele from Solanum habrochaites at marker locus M13 (SEQ ID NO:13); or
(c) further defined as comprised within a plant, plant part, plant cell, or seed.

35. (canceled)

36. The recombinant DNA segment of claim 34, wherein said recombinant DNA segment further comprises an allele from Solanum lycopersicum at marker locus M17 (SEQ ID NO:35).

37. (canceled)

38. A method of producing a tomato plant with improved Tomato Brown Rugose Fruit Virus (ToBRFV) resistance, comprising introgressing into said plant at least one ToBRFV resistance allele within a recombinant chromosomal segment flanked in the genome of said plant by marker locus M12 (SEQ ID NO:12) and marker locus M14 (SEQ ID NO:14) on chromosome 8, wherein said introgressed ToBRFV resistance allele confers to said plant increased resistance to ToBRFV compared to a plant not comprising said allele, and wherein said recombinant chromosomal segment lacks an allele genetically linked thereto that confers an orange mature fruit color phenotype when present, and wherein said introgressing comprises marker-assisted selection.

39. The method of claim 38, wherein:
(a) said introgressing further comprises backcrossing or assaying for said ToBRFV resistance;
(b) said introgressing comprises:
(i) crossing a plant comprising said chromosomal segment with itself or with a tomato plant of a different genotype to produce at least a first progeny plant; and
(ii) selecting a plant comprising said chromosomal segment; or
(c) marker-assisted selection comprises detecting at least a first marker locus genetically linked to said ToBRFV resistance allele selected from the group consisting of: marker locus M12 (SEQ ID NO:6), marker locus M14 (SEQ ID NO:14), marker locus M17 (SEQ ID NO:35), and marker locus M13 (SEQ ID NO:16).

40.-41. (canceled)

42. The method of claim 39, wherein said detecting comprises detecting an allele from Solanum lycopersicum at marker locus M12 (SEQ ID NO:12) and marker locus M14 (SEQ ID NO:14) and an allele from Solanum habrochaites at marker locus M13 (SEQ ID NO:13).

43. The method of claim 42, further comprising detecting an allele from Solanum lycopersicum at marker locus M17 (SEQ ID NO:35).

44. A tomato plant obtainable by the method of claim 38.
